(19) United States
(12) Patent Application Publication
(10) Pub. No.: US 2006/0167020 A1
(43) Pub. Date: Jul. 27, 2006

(54) Pyrazolopyrimidines as kinase inhibitors

(76) Inventors: Scott Howard Dickerson, Durham, NC (US); Dulce Maria Garrido, Durham, NC (US); Wendy Yoon Mills, Durham, NC (US); Kazuya Kano, Tsukuba-shi (JP); Andrew James Peat, Durham, NC (US); Stephen Andrew Thomson, Durham, NC (US); Jayme Lyn Roark Wilson, Durham, NC (US); Hui-Quiang Q Zhou, Durham, NC (US)

Correspondence Address: GLAXOSMITHKLINE;CORPORATE INTELLECTUAL PROPERTY, MAI B475, FIVE MOORE DR., PO BOX 13398, RESEARCH TRIANGLE PARK, NC, 27709-3398, US

(21) Appl. No.: 10/521,909

(22) PCT Filed: Jul. 21, 2003
(86) PCT No.: PCT/US03/22719

§ 371 Date: Jan. 20, 2005

Related U.S. Application Data
(60) Provisional application No. 60/397,947, filed on Jul. 23, 2002.

(51) Int. Cl. A61K 31/519; C07D 487/02

(52) U.S. Cl. 514/262.1; 544/262

(57) ABSTRACT

The present invention relates generally to inhibitors of the kinases and more particularly to novel pyrazolopyrimidine compounds.

### FIELD OF THE INVENTION

The present invention relates generally to inhibitors of the kinases, such as GSK3, and more particularly to novel pyrazolopyrimidine compounds.

### BACKGROUND OF THE INVENTION

The present invention provides compounds that are useful as pharmacological agents for disease states mediated, for example alleviated through the inhibition or antagonism, of protein kinases. In particular, the present invention relates to compounds that demonstrate protein tyrosine kinase and/or protein serine/threonine kinase inhibition.

The protein kinases represent a large family of proteins which play a central role in the regulation of a wide variety of cellular processes and maintaining control over cellular function (Hanks, et al., Science, 1988, 241, 42-52). The loss of control over cellular regulation can often lead to aberrant cell function or death, often resulting in a disease state in the parent organism. A partial list of such kinases includes ab1, ATK, ber-ab1, Blk, Brk, Btk, c-kit, c-met, c-src, CDK1, CDK2, CDK4, CDK6, cRaf1, CSF1R, CSK, EGFR, ErbB2, ErbB3, ErbB4, ERK, Fak, fes, FGFR1, FGFR2, FGFR3, FGFR4, FGFR5, Fgr, FLK-4, flt-1, Fps, Frk, Fyn, GSK3, Hck, IGF-1R, INS-R, Jak, KDR, Lck, Lyn, MEK, p38, PDGFR, PIK, PKC, PYK2, ros, TIE1, TIE2, TRK, Yes, and Zap70. Examples of kinase therapy include, but should not be limited to: (1) inhibition of c-Src (Brickell, Critical Reviews in Oncogenesis 1992, 3, 401-46; Courtneidge, Seminars in Cancer Biology 1994, 5, 239-46), raf (Powis, Pharmacology εt Therapeutics 1994, 62, 57-95) and the cyclin-dependent kinases (CDKs) 1, 2 and 4 in cancer (Pines, Current Opinion in Cell Biology 1992, 4, 144-8; Lees, Current Opinion in Cell Biology 1995, 7, 773-80; Hunter and Pines, Cell 1994, 79, 573-82), (2) inhibition of CDK2 or PDGF-R kinase in restenosis (Buchdunger, et al., Proceedings of the National Academy of Science USA 1995, 92, 2258-62), (3) inhibition of CDK5 and GSK3 kinases for Alzheimer's (Hosoi, et al., Journal of Biochemistry (Tokyo) 1995, 117, 741-9; Aplin, et al., Journal of Neurochemistry 1996, 67, 699-707). (4) inhibition of c-Src kinase in osteoporosis (Tanaka, et al., Nature 1996, 383, 528-31), (5) inhibition of GSK-3 kinase in type-2 diabetes (Borthwick, et al., Biochemical εt Biophysical Research Communications 1995, 210, 738-45), discussed in more detail below; (6) inhibition of the p38 kinase for inflammation (Badger, et al., The Journal of Pharmacology and Experimental Therapeutics 1996, 279, 1453-61); (7) inhibition of VEGF-R 1-3 and TIE-1 and -2 kinases in diseases which involve angiogenesis (Shawver, et al., Drug Discovery Today 1997, 2, 50-63); (8) inhibition of UL97 kinase in viral infections (He, et al., Journal of Virology 1997, 71, 405-11); (9) inhibition of CSF-1R kinase in bone and hematopoetic diseases (Myers, et al., Bioorganic εt Medicinal Chemistry Letters 1997, 7, 421-4), and (10) inhibition of Lck kinase in autoimmune diseases and transplant rejection (Myers, et al., Bioorganic εt Medicinal Chemistry Letters 1997, 7, 417-20).

Inhibitors of certain kinases may also have utility in the treatment of diseases when the kinase is not misregulated, but is nonetheless essential for maintenance of the disease state. In this case, inhibition of the kinase activity would act either as a cure or palliative for these diseases. For example, many viruses, such as human papilloma virus, disrupt the cell cycle and drive cells into the S-phase of the cell cycle (Vousden, FASEB Journal 1993, 7, 872-9). Preventing cells from entering DNA synthesis after viral infection by inhibition of essential S-phase initiating activities such as though kinase inhibition, may disrupt the virus life cycle by preventing virus replication. This same principle may be used to protect normal cells of the body from toxicity of cycle-specific chemotherapeutic agents (Stone, et al., Cancer Research 1996, 56, 3199-202; Kohn, et al., Journal of Cellular Biochemistry 1994, 54, 440-52).

As noted above, GSK3 (glycogen synthase kinase) is identified as a kinase useful in the treatment of type II diabetes. GSK3 inhibits glycogen synthase by direct phosphorylation. Upon insulin signaling, GSK3 is inactivated, thereby allowing the activation of glycogen synthase and possibly other insulin-dependent events.

Type II diabetes, otherwise known as Non-Insulin Dependent Diabetes Mellitus (NIDDM), is initially characterized by decreased sensitivity to insulin (insulin resistance) and a compensatory elevation in circulating insulin concentrations. Increased insulin levels are caused by increased secretion from the pancreatic beta cells in an attempt to overcome the insulin resistance. The resulting hyperinsulinemia is associated with a variety of cardiovascular complications.

As insulin resistance worsens, the demand on the pancreatic beta cells steadily increases until the pancreas can no longer provide adequate levels of insulin, thereby resulting in elevated levels of glucose in the blood. Thus, diabetes causes impaired glucose transport into skeletal muscle and increased hepatic glucose production, in addition to inadequate insulin response. The disorders and conditions associated with hyperglycemia and hyperlipidemia include cardiovascular disease, renal failure, and blindness.

GSK3 inhibition stimulates insulin-dependent processes and is consequently useful in the treatment of diseases and conditions, such as type II diabetes, that are mediated by GSK3 activity, or, more specifically, characterized by a need for the inhibition of GSK3.

For example, Klein et al., PNAS 93:8455-9 (1996) report that lithium ion inhibits GSK3 activity. Lithium has been reported to have anti-diabetic effects such as reduction of plasma glucose levels, increased glycogen uptake, potentiation of insulin, and stimulation of glycogen synthesis in skin, muscle, and fat cells. Lithium, however, effects molecular targets other than GSK3, and is, therefore, not a widely accepted therapy for diabetics.

GSK3 is a proline-directed serinelthreonine kinase. Other examples of GSK3 mediated diseases or conditions include, without limitation, obesity, various CNS disorders such as Alzheimer's Disease, bipolar disorder, and schizophrenia, neurotraumatic injuries such as acute stroke, immune potentiation, baldness or hair loss, atherosclerotic cardiovascular disease, hypertension, polycystic ovary syndrome, ischemia, brain trauma or injury, immunodeficiency, and cancer. See, for example, published PCT application WO 00/38675, the background of which is herein incorporated by reference.

Thus, the compounds of the present invention are believed useful in a variety of disease states, each of which may be characterized as mediated by inhibition or antagonism of protein kinases.

### SUMMARY OF THE INVENTION

The present invention includes compounds of Formula (I) 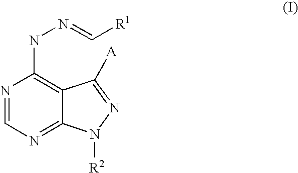
including salts, solvates, and pharmaceutically acceptable derivatives thereof, wherein A is H, alkyl, or aryl; R¹ is D¹, D², D³, D⁴, or D⁵, wherein D¹ is 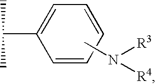
and R³ and R⁴ are each independently H, alkyl, alkylsulfonyl, or —C(O)—(CH₂)ₓ—R⁵, where R⁵ is alkyl, acyl, alkoxy, —(O)—(CH₂)ₓ—(O)-alkyl, or —NR⁶R⁷, where R⁶ and R⁷ are each independently H or alkyl, or R⁶ and R⁷ combine to form a 5- or 6-membered ring, optionally containing one or more additional heteroatoms, optionally containing one or more degrees of unsaturation, and optionally substituted one or more times with alkyl, hydroxy, carboxy, acyl, alkoxy, or halogen, or R³ and R⁴ combine to form a 5- or 6-membered ring, optionally containing one or more additional heteroatoms, optionally containing one or more degrees of unsaturation, and optionally substituted one or more times with alkyl, hydroxy, carboxy, alkoxy, acyl, or halogen;

wherein D² is 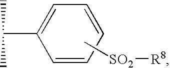
and R⁸ is alkyl, or —NR⁹R¹⁰, where R⁹ and R¹⁰ are each independently selected from H, alkyl, or —(CH₂)ₓ—NR⁶R⁷, where R⁶ and R⁷ are each independently H or alkyl, or R⁶ and R⁷ combine to form a 5- or 6-membered ring, optionally containing one or more additional heteroatoms, optionally containing one or more degrees of unsaturation, and optionally substituted one or more times with alkyl, hydroxy, carboxy, acyl, alkoxy, or halogen;

wherein D³ is 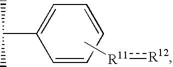
and the dashed line represents an optional double bond; when R¹¹ is —(CH₂)ₓ, the optional dashed double bond does not exist, and R¹² is alkylsulfonyl or —NR¹³R¹⁴, where R¹³ and R¹⁴ are each independently selected from H, alkyl, —(CH₂)ₓ—R¹⁷, where R¹⁷ is alkoxy or —NR¹⁵R¹⁶, where R¹⁵ and R¹⁶ are each independently H or alkyl, or R¹³ and R¹⁴ combine to form a 5- or 6-membered ring, optionally containing one or more additional heteroatoms, optionally containing one or more degrees of unsaturation, and optionally substituted one or more times with alkyl or —(CH₂)ₓ—OH; when R¹¹ is —(CH)—, the optional dashed double bond exists, and R¹² is —(CH)—C(O)—OH;

wherein D⁴ is 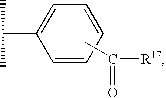
and R¹⁷ is hydroxy, alkoxy, or —NR¹⁸R¹⁹, where R¹⁸ and R¹⁹ are each independently selected from H, alkyl, —(CH₂)ₓ—R² , where R²⁰ is alkylsulfonyl, hydroxy, aryl said aryl optionally substituted with hydroxy or alkoxy, heteroaryl, or —NR²¹R²², where R²¹ and R²² are each independently selected from H, acyl, alkyl, or R²¹ and R²² combine to form a 5- or 6-membered ring, optionally containing one or more additional heteroatoms, optionally containing one or more degrees of unsaturation, and optionally substituted with alkyl or —(CH₂)ₓ—OH; or R¹⁸ and R¹⁹ combine to form a 5- or 6-membered ring, optionally containing one or more additional heteroatoms, optionally containing one or more degrees of unsaturation, and optionally substituted with —(CH₂)ₓ—R²³, where R²³ is alkoxy, hydroxy, —C(O)—R²⁴, where R²⁴ is a 5- or 6-membered ring optionally containing one or more heteroatoms and optionally containing one or more degrees of unsaturation, or —NR²⁵R²⁶, where R²⁵ and R²⁶ are each independently H or alkyl; wherein D⁵ is a 5- or 6-membered ring, optionally containing one or more heteroatoms, optionally containing one or more degrees of unsaturation, optionally fused with an additional 5- or 6-membered ring that optionally contains one or more heteroatoms and optionally contains one or more degrees of unsaturation, wherein the ring or fused ring system may be optionally substituted one or more times with halogen, alkyl, haloalkyl, alkylsulfonyl, alkylthio, hydroxy, alkoxy, oxo, sulfonyl, sulfate ion, nitro, cyano, carboxy, alkoxycarbonyl, aryl where said aryl may be optionally substituted with sulfamoyl, heteroaryl where said heteroaryl may be optionally substituted with alkyl, or —NR²⁷R²⁸, where R²⁷ and R²⁸ are each independently H, alkyl, acyl, alkoxy, alkoxycarbonyl, carboxy, or —(CH₂)ₓ—NR²⁹R³⁰, where R²⁹ and R³⁰ are each independently selected from H and alkyl, or R²⁷ and R²⁸ combine to form a 5- or 6-membered ring, optionally containing one or more additional heteroatoms, optionally containing one or more degrees of unsaturation, and optionally substituted one or more times with alkyl, hydroxy, carboxy, acyl, alkoxy, or halogen, or —(O)_{y}—(CH₂)ₓ—R³¹, where R³¹ is hydroxy, alkoxy, haloalkyl, aryl optionally substituted with halogen, or —NR²⁷R²⁸, where R²⁷ and R²⁸ are as defined above; wherein for each occurrence, x independently is 0, 1, 2, or 3; wherein for each occurrence, y independently is 0 or 1; and R² is heteroaryl substituted one or more times with alkyl, alkoxy, halogen, haloalkyl, haloalkoxy, nitro, or —NR³¹R³², wherein R³¹ and R³² are each independently selected from H and alkyl.

In one embodiment preferably R² is pyridyl. More preferably, R² is pyridyl substituted with alkoxy. More preferably, the alkoxy is methoxy. In one embodiment preferably the pyridyl is 2-pyridyl. In another embodiment preferably the pyridyl is 3-pyridyl. In yet another embodiment, preferably the pyridyl is 4-pyridyl.

In an-other-embodiment preferably R² is thiazolyl.

In another embodiment preferably R² is benzimidazolyl.

In an embodiment of the invention preferably A is H.

Another aspect of the present invention includes pharmaceutical compositions that include a therapeutically effective amount of a compound of the present invention. Preferably such compositions further include one or more of pharmaceutically acceptable carriers, diluents, or excipients.

Another aspect of the present invention includes a method of treating a disorder in a mammal. The disorder is characterized by misregulation of one or more protein kinase. The method includes administering to the mammal a therapeutically effective amount of a compound of the present invention. The kinase may be a serine/threosine kinase. Preferably the kinase is GSK3.

Another aspect of the present invention includes compounds of the present invention for use in therapy. Thus, included are uses of compounds of the present invention in the preparation of a medicament for use in the treatment of a disorder characterized by misregulation of one or more protein kinase. More particularly, the disorder is type 2 diabetes, hyperlipidemia, obesity, CNS disorders, neurotraumatic injuries, immune potentiation, baldness or hair loss, atherosclerotic cardiovascular disease, hypertension, polycystic ovary syndrome, ischemia, immunodeficiency, and cancer.

Another aspect of the present invention includes a method for the treatment or prophylaxis of such disorders through administering a therapeutically effective amount of a compound of the present invention.

Particularly, one aspect of the present invention includes a method of treating type II diabetes by the administration to a mammal (preferably a human) in need thereof of therapeutically effective amounts of a compound of the present invention. Preferably, this aspect includes the administration of at least one additional anti-diabetic agent.

Another aspect of the present invention includes compounds with reference to any of the Examples.

Another aspect of the present invention includes useful intermediates. Thus, the present invention includes compounds of Formula (II): 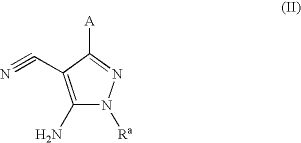
including salts, solvates, and pharmaceutically functional derivatives thereof, where A is H, alkyl, or aryl and R^{a} is heteroaryl substituted one or more times with alkyl, alkoxy, halogen, haloalkyl, haloalkoxy, nitro, or —NR^{b}R^{c}, wherein R^{b} and R^{c} are each independently selected from H and alkyl.

Preferably, R^{a} is selected from 2-pyridyl, thiazolyl, or benzimidazolyl. More preferably, R^{a} is 2-pyridyl substituted with alkoxy. More preferably, the alkoxy is methoxy. Preferably A is H.

The present invention also includes compounds of formula (III) 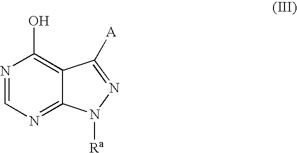
including salts, solvates, and pharmaceutically functional derivatives thereof, where A is H, alkyl, or aryl and R^{a} is heteroaryl substituted one or more times with alkyl, alkoxy, halogen, haloalkyl, haloalkoxy, nitro, or —NR^{b}R^{c}, wherein R^{b} and R^{c} are each independently selected from H and alkyl.

Preferably, R^{a} is selected from pyridyl, thiazolyl, or benzimidazolyl. More preferably R^{a} is pyridyl substituted with alkoxy. More preferably the alkoxy is methoxy. Preferably A is H.

The present invention also includes compounds of formula (IV) 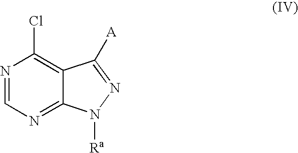
including salts, solvates, and pharmaceutically functional derivatives thereof, where A is H, alkyl, or aryl and R^{a} is heteroaryl substituted one or more times with alkyl, alkoxy, halogen, haloalkyl, haloalkoxy, nitro, or —NR^{b}R^{c}, wherein R^{b} and R^{c} are each independently selected from H and alkyl.

Preferably R^{a} is selected from pyridyl, thiazolyl, or benzimidazolyl. Preferably R^{a} is pyridyl substituted with alkoxy. Preferably the alkoxy is methoxy. Preferably A is H.

Another aspect of the present invention includes compounds of formula (V) 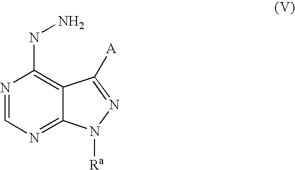
including salts, solvates, and pharmaceutically functional derivatives thereof, where A is H, alkyl, or aryl and R^{a} is heteroaryl substituted one or more times with alkyl, alkoxy, halogen, haloalkyl, haloalkoxy, nitro, or —NR^{b}R^{c}, wherein R^{b} and R^{c} are each independently selected from H and alkyl.

Preferably A is H. Preferably R^{a} is selected from pyridyl, thiazolyl, or benzimidazolyl. More preferably R^{a} is pyridyl substituted with alkoxy. More preferably the alkoxy is methoxy.

### DETAILED DESCRIPTION OF THE PREFERRED EMBODIMENT

The term “alkyl” refers to a straight or branched chain hydrocarbon that may be optionally substituted, with multiple degrees of substitution being allowed. Examples of “alkyl” include, but are not limited to methyl, ethyl, n-propyl, isopropyl, n-butyl, t-butyl, n-pentyl, isobutyl, isopropyl, and the like. The phrase “Cₓ-C_{y}alkyl” refers to an alkyl group, as defined above, containing the specified number of carbon atoms.

The term “alkylene” refers to a straighteor branched chain unsaturated aliphatic hydrocarbon radical that may be optionally substituted, with multiple degrees of substitution being allowed. Examples of “alkylene” include, but are not limited to methylene, ethylene, n-propylene, n-butylene, and the like.

The term “aryl” refers to an optionally substituted benzene ring or to an optionally substituted benzene ring system fused to one or more optionally substituted benzene rings to form, for example, anthracene, phenanthrene, or naphthalene ring systems. Examples of “aryl” groups include, but are not limited to phenyl, 2-naphthyl, 1-naphthyl, biphenyl, as well as substituted derivatives thereof. The term “aralkyl” further refers to groups of —R^{a}R^{b}, where R^{a} is an alkylene as defined herein and R^{b} is an aryl as defined herein. Exemplary “aralkyl” groups include C₁₋₆alkylene-aryl, such as benzyl.

The term “heteroaryl” refers to a monocyclic aromatic ring system, or to a fused bicyclic aromatic ring system comprising two or more aromatic rings. These heteroaryl rings contain one or more nitrogen, sulfur, and/or oxygen atoms, where N-oxides and sulfur oxides and dioxides are permissible heteroatom substitutions and may be optionally substituted, with multiple degrees of substitution being allowed. Examples of “heteroaryl” groups used herein include furan, thiophene, pyrrole, imidazole, pyrazole, triazole, tetrazole, thiazole, oxazole, isoxazole, oxadiazole, thiadiazole, isothiazole, pyridine, pyridazine, pyrazine, pyrimidine, quinoline, isoquinoline, benzofuran, benzothiophene, indole, indazole, and substituted versions thereof. The term “heteroaralkyl” further refers to groups of —RₐR_{b}, where R^{a} is an alkylene as defined herein and R^{b} is a heteroaryl as defined herein.

As used herein, the term “acyl” refers to the group —C(O)Rₐ, where R^{a} is H, alkyl, or aryl. Non-limiting examples of “acyl” groups include formyl, acetyl, benzoyl, and the like.

The term “alkoxy” refers to the group —ORₐ, where R^{a} is alkyl as defined above. Non-limiting examples of “alkoxy” groups include methoxy, ethoxy, and the like.

As used herein, the term “oxo” refers to the group ═O.

As used herein, the term “hydroxy” refers to the group —OH.

As used herein, the term “carboxy” refers to the group —COOH.

The term “halogen” refers to fluorine, chlorine, bromine, or iodine.

The term “haloalkyl” refers to an alkyl group, as defined herein, that is substituted with at least one halogen. Non-limiting examples of “haloalkyl” groups include methyl, ethyl, propyl, isopropyl, n-butyl, and t-butyl substituted independently with one or more halogens, e.g., fluoro, chloro, bromo, and/or iodo. The term “haloalkyl” should be interpreted to include such substituents as perfluoroalkyl and the like.

The term “haloalkoxy” refers to the group —ORₐ, where R^{a} is haloalkyl as defined above.

As used herein, the term “sulfonyl” shall refer to the group —S(O)₂—.

As used herein, the term “alkylsulfonyl” refers to the group —S(O)₂Rₐ, where Rₐ is alkyl as defined above.

As used herein, the term “alkylthio” refers to the group —SRₐ, where R^{a} is alkyl as defined above.

As used herein, the term “sulfamoyl” refers to a group —SO₂—NH₂.

As used herein, the term “carbamoyl” refers to the group —C(O)NH₂.

As used herein, the term “carboxamide” refers to the group —C(O)N(Rₐ)₂, where R^{a} is alkyl or aryl as defined herein.

As used herein, the term “alkoxycarbonyl” refers to the group —C(O)ORₐ, where Rₐ is alkyl or aryl as defined herein.

The compounds of the present invention may have the ability to crystallize in more than one form, a characteristic known as polymorphism. Such polymorphic forms (“polymorphs”) are within the scope of the present invention. Polymorphism generally can occur as a response to changes in temperature or pressure, or both, and can also result from variations in the crystallization process. Polymorphs can be distinguished by various physical characteristics that are known in the art such as x-ray diffraction patterns, solubility, and melting point.

Certain of the compounds described herein contain one or more chiral centers, or may otherwise be capable of existing as multiple stereoisomers. The scope of the present invention includes mixtures of stereoisomers as well as purified enantiomers, or enantiomerically or diastereomerically enriched mixtures. Also included within the scope of the invention are the individual isomers of the compounds, as well as any wholly or partially equilibrated mixtures thereof. The present invention also covers the individual isomers of the compounds represented by the formulas above as mixtures with isomers thereof in which one or more chiral centers are inverted.

As noted above, the present invention includes salts, solvates, and pharmaceutically functional derivatives of the compounds of the present invention. Salts include addition salts, metal salts, or optionally alkylated ammonium salts. Examples of such salts include hydrochloric, hydrobromic, hydroiodic, phosphoric, sulfuric, trifluoroacetic, trichloroacetic, oxalic, maleic, pyruvic, malonic, succinic, citric, mandelic, benzoic, cinnamic, methane sulphonic, ethane sulphonic, picric, and the like. Further salts include lithium, sodium, potassium, magnesium, and the like. Reference is also made to Journal of Pharmaceutical Science, 1997, 66, 2, incorporated herein by reference, as relevant to salts.

As used herein, the term “solvate” refers to a complex of variable stoichiometry formed by a solute or a salt or pharmaceutically functional derivative thereof and a solvent. Such solvents for the purpose of the invention should not interfere with the biological activity of the solute. Examples of solvents include, but are not limited to water, methanol, ethanol, and acetic acid. Preferably the solvent used is a pharmaceutically acceptable solvent. Examples of pharmaceutically acceptable solvents include water, ethanol, and acetic acid.

The term “pharmaceutically functional derivative” refers to any pharmaceutically acceptable derivative of a compound of the present invention, for example, an ester or an amide, which upon administration to a mammal is capable of providing (directly or indirectly) a compound of the present invention or an active metabolite thereof. Such derivatives are recognizable to those skilled in the art, without undue experimentation. Nevertheless reference is made to the teaching of Burger's Medicinal Chemistry and Drug Discovery, 5^{th} Edition, Vol 1: Principles and Practice, which is incorporated herein by reference to the extent of teaching pharmaceutically functional derivatives.

While it is possible that compounds of the present invention may be administered as the raw chemical, preferably the compounds of the present invention are presented as an active ingredient within a pharmaceutical formulation, as are known in the art. Accordingly, the present invention further includes a pharmaceutical formulation comprising a compound of the present invention, or salt, solvate, or functional derivative thereof together with one or more pharmaceutically acceptable carriers. Optionally, other therapeutic and/or prophylactic ingredients may be included in the pharmaceutical formulation. For example, the compounds of the present invention may be combined with other agents, such as, without limitation, one or more other anti-diabetic agent such as insulin, alpha glucosidase inhibitors, biguanides, insulin secretagogues such as sulphonylureas, insulin senstizers such as thiazolidinediones, and/or dipeptidyl peptidase inhibitors.

Formulations of the present invention include those especially formulated for oral, buccal, parental, transdermal, inhalation, intranasal, transmucosal, implant, or rectal administration. Among the variety of administrations, oral administration typically is preferred. For oral administration tablets, capsules, and caplets may contain conventional excipients such as binding agents, fillers, lubricants, disintegrants, and/or wetting agents. Non-limiting examples of binding agents include syrup, acacia, gelatin, sorbitol, tragacanth, mucilage of starch, or polyvinylpyrrolidone (PVP). Non-limiting examples of fillers include, for example, lactose, sugar, microcrystalline cellulose, maize-starch, calcium phosphate or sorbitol. Non-limiting examples of lubricants include, for example, magnesium sterate, stearic acid, talc, polyethylene glycol or silica. Non-limiting examples of disintegrants include, for example, potato starch or sodium starch glycollate. A non-limiting example of a wetting agent includes sodium lauryl sulfate. The tablets additionally may be coated according to methods known in the art.

Alternatively, the compounds of the present invention may be incorporated into oral liquid preparations such as aqueous or oily suspensions, solutions, emulsions, syrups, or elixirs. Moreover, formulations containing these compounds may be presented as a dry product for constitution with water or other suitable vehicle before use. Liquid preparations may contain conventional additives. Non-limiting examples of such additives include suspending agents such as sorbitol syrup, methyl cellulose, glucose/sugar syrup, gelatin, hydroxyethylcellulose, carboxymethyl cellulose, aluminum sterate gel or hydrogenated edible fats. Additionally, emulsifying agents such as lecithin, sorbitan mono-oleate or acacia; non-aqueous vehicles (which may include edible oils) such as almond oil, fractionated coconut oil, oily esters, propylene glycol or ethyl alcohol may be included. Further, preservatives such as methyl or propyl p-hydroxybenzoates or sorbic acid, may be incorporated into the preparation. Such preparations may also be formulated as suppositories, for example, containing conventional suppository bases such as cocoa butter or other glycerides.

Additionally, formulations of the present invention may be formulated for parenteral administration by injection or continuous infusion. Formulations for injection may take such forms as suspensions, solutions, or emulsions in oily or aqueous vehicles, and may contain formulatory agents such as suspending, stabilizing and/or dispersing agents. Alternatively, the active ingredient may be in powder form for constitution with a suitable vehicle, for example, sterile, pyrogen-free water, before use.

The formulations according to the invention may also be formulated as a depot preparation. Such long acting formulations may be administered by implantation, for example, subcutaneously or intramuscularly, or by intramuscular injection. Accordingly, the compounds of the invention may be formulated with suitable polymeric or hydrophobic materials, such as an emulsion in an acceptable oil, ion exchange resins, or as sparingly soluble derivatives, such as a sparingly soluble salt.

Pharmaceutical formulations may be presented in unit dose forms containing a predetermined amount of active ingredient per unit dose. Such a unit may contain certain amounts of a compound of the present invention depending on the condition being treated, the route of administration, and the age, weight and condition of the patient. Preferred unit dosage formulations are those containing a predetermined dose, such as a daily dose, or an appropriate fraction thereof, of an active ingredient. Such pharmaceutical formulations may be prepared by any of the methods well known in the pharmacy art.

As used herein, the term “effective amount” means that amount of a drug or pharmaceutical agent that will elicit the biological or medical response of a tissue, system, animal, or human that is being sought, for instance, by a researcher or clinician. Furthermore, the term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

A “therapeutically effective amount” of a compound of the present invention will depend upon a number of factors including, for example, the age and weight of the animal, the precise condition requiring treatment and its severity, the nature of the formulation, and the route of administration. Therapeutic effectiveness ultimately will be at the discretion of the attendant physician or veterinarian. An effective amount of a salt or solvate, or pharmaceutically functional derivative thereof, may be determined as a proportion of the effective amount of a compound of the present invention per se.

Experimentals

The following examples illustrate aspects of this invention, but should not be construed as limitations. Unless otherwise noted, all starting materials were obtained from commercial suppliers or obtained through synthetic methods known to those skilled in the art. As used herein the symbols and conventions used in these processes, schemes and examples are consistent with those used in the contemporary scientific literature, for example, the Journal of the American Chemical Society or the Journal of Biological Chemistry. Specifically, the following abbreviations may be used in the examples and throughout the specification:

| | |
|---|---|
| g (grams); | mg (milligrams); |
| L (liters); | mL (milliliters); |
| μL (microliters); | psi (pounds per square inch); |
| M (molar); | mM (millimolar); |
| i.v. (intravenous); | Hz (Hertz); |
| MHz (megahertz); | mol (moles); |
| mmol (millimoles); | RT (room temperature); |
| min (minutes); | h (hours); |
| mp (melting point); | TLC (thin layer chroma- |
| Tᵣ (retention time); | tography); |
| MeOH (methanol); | RP (reverse phase); |
| TEA (triethylamine); | I-PrOH (isopropanol); |
| TFAA (trifluoroacetic anhydride); | TFA (trifluoroacetic acid); |
| DMSO (dimethylsulfoxide); | THF (tetrahydrofuran); |
| DCE (dichloroethane); | EtOAc (ethyl acetate); |
| HOAc (acetic acid); | DMF (N,N-dimethylformamide); |
| mCPBA (meta-chloroperbenzoic acid; | EDC (ethylcarbodiimide |
| BOC (tert-butyloxycarbonyl); | hydrochloride); |
| DCC (dicyclohexylcarbodiimide); | CBZ (benzyloxycarbonyl); |
| Ac (acetyl); | Me (methyl); |
| TMSE (2-(trimethylsilyl)ethyl); | atm (atmosphere); |
| TIPS (triisopropylsilyl); | TMS (trimethylsilyl); |
| DMAP (4-dimethylaminopyridine); | TBS (t-butyldimethylsilyl); |
| HPLC (high pressure liquid | tBu (tert-butyl). |
| chromatography); | |
| Et (ethyl); | |

All references to ether are to diethyl ether; brine refers to a saturated aqueous solution of NaCl. Unless otherwise indicated, all temperatures are expressed in ° C. (degrees Centigrade). All reactions were conducted under an inert atmosphere at room temperature unless otherwise noted.

¹H NMR spectra were recorded on a Varian VXR-300, a Varian Unity-300, a Varian Unity-400 instrument, or a General Electric QE-30Q. Chemical shifts are expressed in parts per million (ppm, δ units). Coupling constants are in units of hertz (Hz). Splitting patterns describe apparent multiplicities and are designated as s (singlet), d (doublet), t (triplet), q (quartet), m (multiplet), br (broad).

Low-resolution mass spectra (MS) were recorded on a JOEL JMS-AX505HA, JOEL SX-102, or a SCIEX-APliii spectrometer; high resolution MS were obtained using a JOEL SX-102A spectrometer. All mass spectra were taken under electrospray ionization (ESI), chemical ionization (CI), electron impact (EI) or by fast atom bombardment (FAB) methods. Infrared (IR) spectra were obtained on a Nicolet 510 FT-IR spectrometer using a 1-mm NaCl cell. All reactions were monitored by thin-layer chromatography on 0.25 mm E. Merck silica gel plates (60F-254), visualized with UV light, 5% ethanolic phosphomolybdic acid or p-anisaldehyde solution. Flash column chromatography was performed on silica gel (230-400 mesh, Merck). Optical rotations were obtained using a Perkin Elmer Model 241 Polarimeter. Melting points were determined using a Mel-Temp II apparatus and are uncorrected.

IUPAC names are included to further identify particular compounds of the present invention. The IUPAC names stated herein should in no way limit the scope of the present invention. 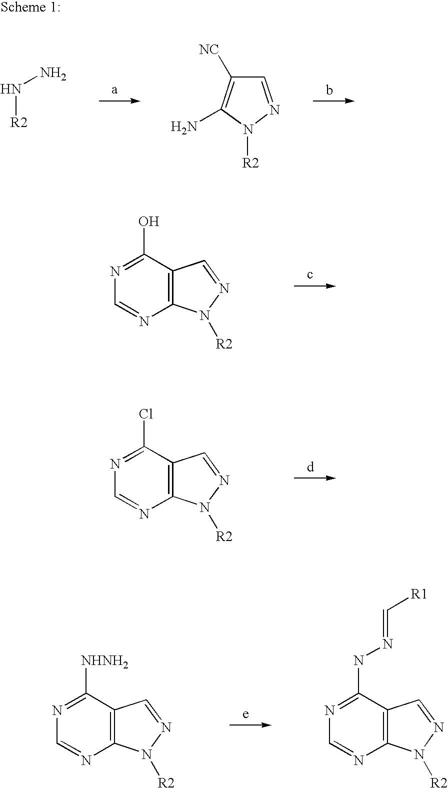
a: ethoxymethylenemalonitrile (1 eq), triethylamine (1.2 eq), ethanol; b: formic acid; c: phosphorus oxychloride; d: hydrazine hydrate (6 eq), ethanol; e: appropriate aldehyde (1 eq), pyrrolidine (cat.), ethanol. 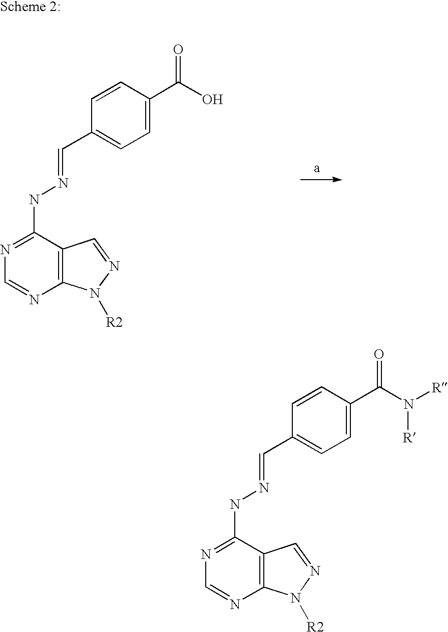
a: appropriate amine (1.5 eq), diethyleyanophosphonate (2 eq), triethylamine (3 eq), DMF 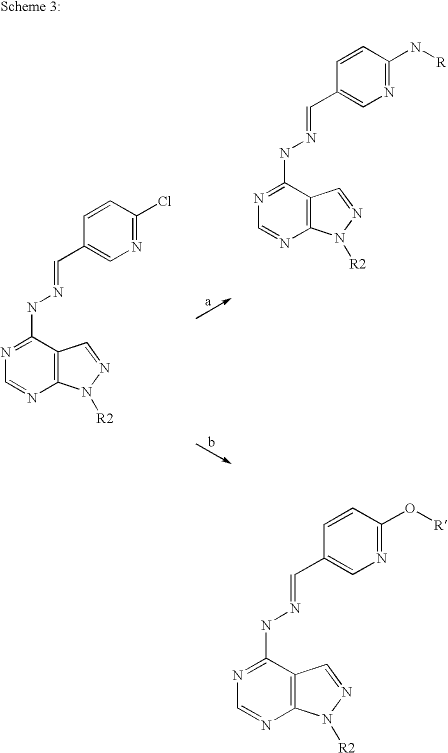
a: appropriate amine, diisopropylethylamine.b:i:Sodium hydride (12 eq), appropriate alcohol (18 eq), THF ii: DMSO

### INTERMEDIATES EXAMPLE A

### 4-Hydrazino-1-(6-methoxypyridin-2-yl)-1H-pyrazolo[3,4-d]pyrimidine

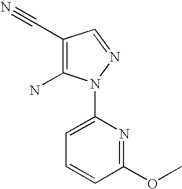

### a. 5-Amino-1-(6-methoxypyridin-2-yl)-1H-pyrazole-4-carbonitrile

To a solution of 2-hydrazino-6-methoxypyridine hydrochoride (1.46 g, 8.41 mmol) in 25 mL of ethanol was added 2-(ethoxymethylene)malononitrile (1.03 g, 8.41 mmol) and triethylamine (1.3 mL, 9.25 mmol). Mixture was refluxed for ca. 7 h, concentrated under reduced pressure and the resulting solid was triturated with aqueous sodium bicarbonate to give the product as a yellow solid (0.55 g, 30%).

¹H NMR (DMSO) δ 7.90 (m, 2H), 7.83 (s, 2H), 7.38 (d, 1H), 6.78 (d, 1H), 3.90 (s, 3H) ppm.

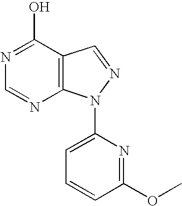

### b. 1-(6-Methoxypyridin-2-yl)-1H-pyrazolo[3,4-d]pyrimidin-4-ol

5-amino-1-(6-methoxypyridin-2-yl)-1H-pyrazole-4-carbonitrile (a, above) (0.53 g, 2.44 mmol) was dissolved in 40 mL of formic acid and refluxed for ca. 24 h. The mixture was cooled to RT, concentrated under reduced pressure and diluted with ether. The resulting solid was collected by filtration and washed with ether to give the product as a white solid (0.39 g, 70%).

¹H NMR (DMSO) δ 12.47 (s, 1H), 8.33 (s, 1H), 8.18 (d, 1H), 7.94 (t, 1H), 7.51 (d, 1H), 6.90 (d, 1H), 3.90 (s, 3H) ppm.

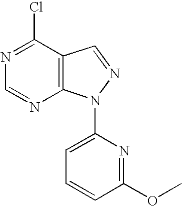

### c. 4-Chloro-1-(6-methoxypyridin-2-yl)-1H-pyrazolo[3,4-d]pyrimidine

1-(6-methoxypyridin-2-yl)-1H-pyrazolo[3,4-d]pyrimidin-4-ol (b, above) (0.38 g, 1.65 mmol) was dissolved in phosphorous oxychloride (8 mL) and 2-3 drops of DMF was added. The mixture was heated at reflux for ca. 5 h. The mixture was concentrated under reduced pressure and quenched into an ice sodium bicarbonate mixture and extracted with methylene chloride. The organic phase was washed with aqueous sodium bicarbonate and concentrated to give the product as a white solid (0.34 g, 80%).

¹H NMR (DMSO) δ 8.99 (s, 1H), 8.76 (s, 1H), 7.99 (t, 1H), 7.66 (d, 1H), 6.63 (d, 1H), 3.94 (s, 3H) ppm.

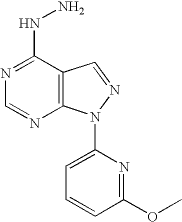

### d. 4-Hydrazino-1-(6-methoxypyridin-2-yl)-1H-pyrazolo[3,4-d]pyrimidine

4-Chloro-1-(6-methoxypyridin-2-yl)-1H-pyrazolo[3,4-d]pyrimidine (c, above) (0.33 g, 1.26 mmol) was dissolved in ethanol (65 mL) and hydrazine mono-hydrate (0.37 mL, 7.6 mmol) was added. The mixture was heated at 55° C. for ca. 18 h and concentrated under reduced pressure. The resulting solid was triturated with aqueous sodium bicarbonate to give the product as a white solid (0.29 g, 90%).

ES-MS m/z 258 (MH⁺).

### INTERMEDIATES EXAMPLE B

### 4-Hydrazino-1-pyridin-2-yl-1H-pyrazolo[3,4-d]pyrimidine

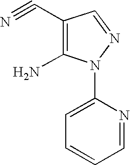

### a. 5-Amino-1-pyridin-2-yl-1H-pyrazole-4-carbonitrile

To 2-hydrazinopyridine (3.00 g, 27.5 mmol) in 50 mL of ethanol was added 2-(ethoxymethylene)malononitrile (3.37 g, 27.5 mmol) and triethylamine (3.8 mL,27.5 mmol). Mixture was refluxed of ca. 3.5 h. After cooling to RT the resulting solids were collected to give the product as a white solid (4.33 g, 85%).

¹H NMR (DMSO) δ 8.46 (dd, 1H), 8.11 (brs, 2H), 8.01 (m, 1H), 7.88 (s, 1H), 7.84 (d, 1H), 7.35 (m, 1H) ppm.

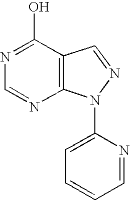

### b. 1-Pyridin-2-yl-1H-pyrazolo[3,4-d]pyrimidin-4-ol

5-amino-1-pyridin-2-yl-1H-pyrazole-4-carbonitrile (b, above) (4.30 g, 23.2 mmol) was dissolved in 60 mL of formic acid and refluxed for ca. 22 h. The mixture was cooled to RT, concentrated under reduced pressure and diluted with ether. The resulting solid was collected by filtration and washed with ether to give the product as a white solid (4.71 g, 95%).

¹H NMR (DMSO) δ 12.40 (brs, 1H), 8.61 (dd, 1H), 8.34 (s, 1H), 8.17 (s, 1H), 8.07 (dt, 1H), 7.91 (d, 1H), 7.49 (dd, 1H) ppm.

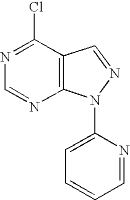

### c. 4-Chloro-1-pyridin-2-yl-1H-pyrazolo[3,4-d]pyrimidine

1-pyridin-2-yl-1H-pyrazolo[3,4-d]pyrimidin-4-ol (b, above) (4.70 g, 22.1 mmol) was dissolved in phosphorous oxychloride (40 mL) and 2-3 drops of DMF was added. The mixture was heated at reflux for ca. 4.5 h. The mixture was concentrated under reduced pressure and quenched into an ice sodium bicarbonate mixture and extracted with methylene chloride. The organic phase was washed with aqueous sodium bicarbonate and concentrated to give the product as a white solid (3.1 g, 61%).

¹H NMR (DMSO) δ 8.99 (s 1H), 8.78 (s, 1H), 8.66 (d, 1H), 8.12 (dt, 1H), 8.06 (d, 1H), 7.53 (t, 1H) ppm.

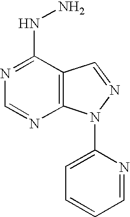

### d. 4-Hydrazino-1-pyridin-2-yl-1H-pyrazolo[3,4-d]pyrimidine

4-chloro-1-pyridin-2-yl-1H-pyrazolo[3,4-d]pyrimidine (c, above) (3.10 g, 13.4 mmol) was dissolved in ethanol (200 mL) and hydrazine mono-hydrate (3.9 mL, 80.3 mmol) was added. The mixture was heated at 50° C. for ca. 24 h and concentrated under reduced pressure. The resulting solid was triturated with aqueous sodium bicarbonate to give the product as a white solid (2.76 g, 91%).

ES-MS m/z 228 (MH⁺).

### INTERMEDIATES EXAMPLE C

### 4-Hydrazino-1-(1,3-thiazol-2-yl)-1H-pyrazolo[3,4-d]pyrimidine

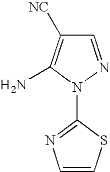

### a. 5-Amino-1-(1,3-thiazol-2-yl)-1H-pyrazole-4-carbonitrile

Ethoxymethylenemalononitrile (0.290 g, 2.38 mmol) was added to a solution of 2-hydrazino-1,3-thiazole (0.274 g, 2.38 mmol) in 10 mL of absolute ethanol. The mixture was heated at reflux for 2 hours. After cooling to room temperature, the solvent was evaporated under vacuum to give 0.448 g (98%) of a tan solid.

¹H NMR (DMSO) δ 8.00 (br s, 2H), 7.95 (s, 1H), 7.65 (d, 1H), 7.55 (d, 1H) ppm.

ES-MS m/z 192 (MH⁺)

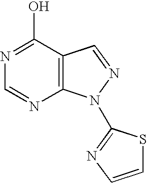

### b. 1-(1,3-Thiazol-2-yl)-1H-pyrazolo[3,4-d]pyrimidin-4-ol

A mixture of 5-amino-1-(1,3-thiazol-2-yl)-1H-pyrazole-4-carbonitrile (a, above) (0.445 g, 2.33 mmol) and 10 mL of 88% formic acid was heated at 100° C. for 18 hours. After cooling to room temperature, diethyl ether was added and the precipitated solid was collected by filtration, washed with ether and dried under vacuum to give 0.350 g (69%) of product as a white solid.

ES-MS m/z 220 (MH⁺)

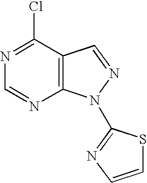

### c. 4-Chloro-1-(1,3-thiazol-2-yl)-1H-pyrazolo[3,4-d]pyrimidine

A suspension of 1-(1,3-thiazol-2-yl)-1H-pyrazolo[3,4-d]pyrimidin-4-ol (b, above) (0.290 g, 1.32 mmol) in 2 mL of phosphorus oxychloride was heated to 100° C. for 1 hour. After cooling to room temperature the mixture was poured into ice and extracted with dichloromethane. The organic phase was dried over anhydrous sodium sulfate and the solvent removed under vacuum to give 0.267 g (85%) of product as a pale yellow solid.

¹H NMR (DMSO) δ 9.15 (s, 1H), 8.90 (s, 1H), 7.85 (dd, 2H) ppm. ES-MS m/z 238 (MH⁺)

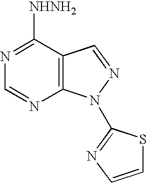

### d. 4-Hydrazino-1-(1,3-thiazol-2-yl)-1H-pyrazolo[3,4-d]pyrimidine

Hydrazine monohydrate (0.32 mL, 6.72 mmol) was added to a suspension of 4-chloro-1-(1,3-thiazol-2-yl)-1H-pyrazolo[3,4-d]pyrimidinee (c above) (0.266 g, 1.12 mmol) in 10 mL of absolute ethanol. The mixture was heated at reflux for 1 hour. The solvent was evaporated and the solid residue was suspended in saturated aqueous sodium bicarbonate, filtered, washed with water, and dried under vacuum to yield 0.159 g (61%) of product as a beige solid.

ES-MS m/z 238 (MH⁺)

### INTERMEDIATES EXAMPLE D

### 4-Hydrazino-1-pyridin-4-yl-1H-pyrazolo[3,4-d]pyrimidine

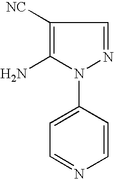

### a. 5-Amino-1-pyridin-4-yl-1H-pyrazole-4-carbonitrile

To a suspension of 4-hydrazinopyridine hydrochloride (2.5 g, 17.2 mmol) in ethanol (100 ml) were added 2-ethoxymethylenemalononitrile (2.6 g, 21.3 mmol) and triethylamine (3.2 ml, 23 mmol). The mixture was refluxed for 30 min, concentrated and purified by column chromatography with ethyl acetate to give clean product as a solid (2.0 g, 63% yield).

¹H NMR(300 MHz, DMSO) δ 8.71 (d, 2H), 7.93 (s, 1H), 7.63 (d, 2H), 7.10 (s, 2H) ppm;

ES-MS m/z 186 (MH⁺).

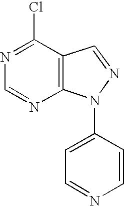

### b. 4-Chloro-1-pyridin-4-yl-1H-pyrazolo[3,4-d]pyrimidine

A solution of 5-amino-1-pyridin-4-yl-1H-pyrazole-4-carbonitrile (a, above) (2.0 g, 11 mmol) in 88% formic acid (50 mL) was stirred at 100° C. overnight. The solution was concentrated to 10 mL and ether was added. Solid was filtered, washed with ether (2×) and dried under vacuum. The resulting solid was dissolved in phosphorus oxytrichloride (30 mL) and the mixture was refluxed for 4 hours. Excess POCl₃ was removed under vacuum and ice water was added. The mixture was neutralized with 5N sodium hydroxide solution and extracted with dichloromethane three times. The combined organic layers were washed with brine three times and dried over magnesium sulfate. Removal of solvent and purification by column chromatography with hexane:ethyl acetate (1:1) gave product as a solid (1.3 g, 52%).

¹H NMR(300 MHz, DMSO) δ 9.12 (s, 1H), 8.92 (s, 1H), 8.81 (d, 2H), 8.36 (d, 2H) ppm;

ES-MS m/z 232 (MH⁺).

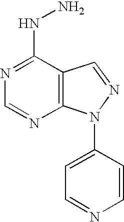

### c. 4-Hydrazino-1-pyridin-4-yl-1H-pyrazolo[3,4-d]pyrimidine

To a suspension of 4-chloro-1-pyridin-4-yl-1H-pyrazolo[3,4-d]pyrimidine (b, above) (780 mg, 3.34 mmol) in ethanol (60 mL) was added hydrazine (0.52 mL, 16.8 mmol). The mixture was refluxed for 4 hours. Solid was filtered, washed with ethanol twice and dried under vacuum to give product as a solid (600 mg, 78%).

¹H NMR (300 MHz, DMSO) δ 9.5 (br, 1H), 8.7 (br, 3H), 8.4 (br, 3H), 5.0 (br, 2H) ppm;

ES-MS m/z 228 (MH⁺).

### INTERMEDIATES EXAMPLE E

### 1-(1H-Benzimidazol-4-yl)-4-hydrazino-1H-pyrazolo[3,4-d]pyrimidine

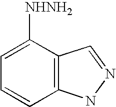

### a. 4-Hydrazino-1H-benzimidazole

A solution of 1H-benzimidazol-4-amine (0.360 g, 2.70 mmol) in 5 mL of anhydrous tetrahydrofuran was cooled to 0° C. Boron trifluoride etherate (0.50 mL, 4.06 mmol) was added dropwise and the mixture was stirred at 0° C. for 20 minutes. A solution of isoamyl nitrite (0.43 mL, 3.24 mmol) in 2 mL anhydrous tetrahydrofuran was added and stirring was continued for 30 minutes. Hexane (10 mL) was added and the precipitated solid was collected by filtration and added in portions to a cold (0° C.), stirred solution of tin (II) chloride (1.54 g, 8.1 mmol) in 2 mL of concentrated hydrochloric acid. The reaction mixture was allowed to warm to room temperature overnight then made alkaline with 50% aqueous sodium hydroxide. Ethyl acetate (50 mL) was added and the mixture was filtered through Celite. The organic phase was dried over anhydrous sodium sulfate and the solvent was removed under vacuum to give 0.262 g of a tan solid. The product was dissolved in methanol and 3.0 mL of 1.0 M ethereal hydrogen chloride was added. The precipitated solid was collected by filtration, washed with diethyl ether and dried under vacuum to give 0.294 g (59%) of the hydrochloride salt as a tan solid.

¹H NMR (DMSO) δ 8.00 (s, 1H), 7.00 (t, 1H), 6.80 (d, 1H), 6.65 (d, 1H), 6.50 (s, 1H), 5.15 (br s, 1H), 4.40 (br s, 2H) ppm; ES-MS m/z 149 (MH⁺).

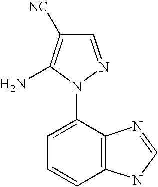

### b. 5-Amino-1-(1H-benzimidazol-4-yl)-1H-pyrazole-4-carbonitrile

4-Hydrazino-1H-benzimidazole hydrochloride (a, above) (0.194 g, 1.05 mmol) was treated with ethoxymethylenemalononitrile (0.128 g, 1.05 mmol) and triethylamine (0.17 mL, 1.26 mmol) in absolute ethanol as described for 5-Amino-1-(4-methoxyphenyl)-1H-pyrazole-4-carbonitrile Flash chromatography (silica gel, 1-5% methanol in dichloromethane) gave 0.136 g (58%) of product as a yellow solid.

¹H NMR (DMSO) δ 13.0 (br s, 1H), 8.40 (s, 1H), 7.88 (s, 1H), 7.64 (d, 1H), 7.40 (d, 1H), 7.34 (t, 1H), 7.20 (br s, 2H) ppm; ES-MS m/z 225 (MH⁺). 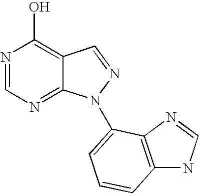
c. 1-(1H-Benzimidazol-4-yl)-1H-pyrazolo[3,4-d]pyrimidin-4-ol

5-Amino-1-(1H-benzimidazol-4-yl)-1H-pyrazole-4-carbonitrile (b, above) (0.133 g, 0.59 mmol) was treated with formic acid as described for 1-(3-methylphenyl)-1H-pyrazolo[3,4-s]pyrimidin-4-ol to give 0.143 g of product as an off-white solid.

¹H NMR (DMSO) δ 12.8 (br s, 1H), 12.45 (br s, 1H), 8.45 (s, 1H), 8.30 (s, 1H), 8.20 (s, 2H), 7.80 (d, 1H), 7.40 (t, 1H) ppm; ES-MS m/z 253 (MH⁺).

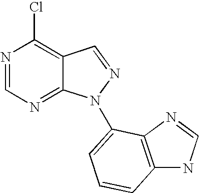

### d. 1-(1H-Benzimidazol-4-yl)-4-chloro-1H-pyrazolo[3,4-d]pyrimidine

A suspension of 1-(1H-benzimidazol-4-yl)-1H-pyrazolo[3,4-d]pyrimidin-4-ol (c, above) (0.533 g, 2.11 mmol) in 8 mL of phosphorus oxychloride was heated to 100° C. for 18 hours. The reaction mixture was cooled to room temperature and the excess phosphorus oxychloride was removed under vacuum. The residue was taken up in dichloromethane and added to ice cold saturated aqueous sodium bicarbonate. The organic phase was filtered, dried over sodium sulfate and evaporated to give the product as a yellow solid (34 mg, 6%).

¹H NMR (DMSO) δ 12.8 (br s, 1H), 8.95 (s, 1H), 8.85 (s, 1H), 8.30 (s, 1H), 7.85 (d, 1H), 7.70 (m, 1H), 7.45 (t, 1H) ppm; ES-MS m/z 271 (MH⁺).

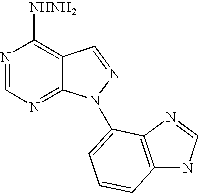

### e. 1-(1H-Benzimidazol-4-yl)-4-hydrazino-1H-pyrazolo[3,4-d]pyrimidine

Hydrazine hydrate (0.036 mL, 0.75 mmol) was added to a solution of 1-(1H-benzimidazol-4-yl)-4-chloro-1H-pyrazolo[3,4-d]pyrimidine (d, above) (0.034 g, 0.125 mmol) in 2 mL of absolute ethanol. The mixture was heated at reflux for 2 hours, cooled to room temperature and the solvent was removed under vacuum. The residue was suspended in saturated aqueous sodium bicarbonate, stirred for 10 minutes, filtered, washed with water and dried under vacuum to give 25 mg (75%) of product as a white solid.

ES-MS m/z 267 (MH⁺).

or

### e. 1-(1H-Benzimidazol-4-yl)-4-hydrazino-1H-pyrazolo[3,4-d]pyrimidine

A suspension of 5-amino-1-(1H-benzimidazol-4-yl)-1H-pyrazole-4-carbonitrile (0.97 g, 4.33 mmol) in 10 mL of trimethyl orthoformate was heated to 100° C. under a reflux condenser, under nitrogen, for 18 hours. After cooling to room temperature the excess trimethyl orthoformate was removed in vacuo and the residue was triturated with diethyl ether. A portion of the resulting solid (0.250 g. 0.94 mmol) was treated with hydrazine (0.27 mL, 5.63 mmol) in 2 mL of ethanol at room temperature for 18 hours. The reaction mixture was filtered and the crude solid product was washed with ethanol, water and dried under vacuum to give 73 mg of product as an off-white solid.

ES-MS m/z 267 (MH⁺).

### INTERMEDIATES EXAMPLE F

### N-[2-(dimethylamino)ethyl]-4-formylbenzenesulfonamide

### a. 4-eyano-N-[2-(dimethylamino)ethyl]benzenesulfonamide

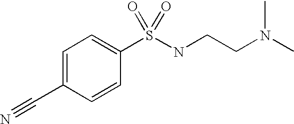

(U16945/187/1)

N,N-dimethylethylenediamine (3.40 mL; 31.10 mmol) was added to a solution of 4-cyanobenzenesulfonyl chloride (2.50 g; 12.40 mmol) in THF (25 mL) at RT. After 16h, saturated NaHCO₃ (100 mL) and ethylacetate (250 mL) were added. The organic layer was separated, dried over Na₂SO₄, filtered and concentrated to give the title compound (3.00 g; 96%).

¹H NMR (300 MHz, CDCl₃) δ7.98 (s, 2H), 7.80 (d, 2H), 5.25 (s br, 1H), 2.98 (t, 2H), 2.33 (t, 2H), 2.07 (s, 6H).

### b. N-[2-(dimethylamino)ethyl]-4-formylbenzenesulfonamide

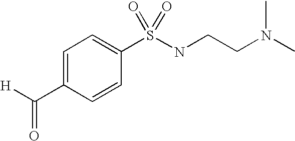

(U16945/18712)

A solution of 1 M diisobutylaluminum hydride in hexanes (9.57 mL; 9.57 mmol) was added slowly to a solution of 4-cyano-N-[2-(dimethylamino)ethyl]benzenesulfonamide (a, above) (1.10 g; 4.35 mmol) in toluene (50 mL) at RT under N₂. After 3 h, an aqueous solution of 5% H₂SO₄ (50 mL) was added and the mixture was stirred for 1 h. Saturated NaHCO₃ (100 mL) and ethylacetate (200 mL) were added. The aqueous layer was separated and washed with ethylacetate. The combined organic layers were dried over Na₂SO₄, filtered and concentrated to give the title compound (0.89 g; 80%).

¹H NMR (300 MHz, CDCl3) δ10.09 (s, 1H), 8.04-7.99 (m, 4H),4.96 (s br, 1H), 2.99 (t, 2H), 2.32 (t, 2H), 2.06 (s, 6H).

### INTERMEDIATES EXAMPLE G

### 4-Hydrazino-1-pyridin-3-yl-1H-pyrazolo[3,4-d[pyrimidine

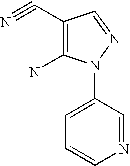

### a. 5-Amino-1-pyridin-3-yl-1H-pyrazole-4-carbonitrile

To 3-hydrazinopyridine hydrochloride (0.35 g, 2.4 mmol) in 25 mL of ethanol was added 2-(ethoxymethylene)malononitrile (0.31 g, 2.5 mmol) and triethylamine (0.7 mL, 4.9 mmol). Mixture was refluxed of ca. 6 h. After cooling to RT the resulting solids were collected, titurate with saturated bicarbonate, to give the product as a tan solid (0.30 g, 68%).

¹H NMR (DMSO) δ 8.72 (d,₁H),8.61 (d,₁H),7.93 (dt,1H),7.84 (s,₁H),7.55 (dd, 1 H), 6.88 (s, 2H) ppm; ES-MS m/z 186 (MH⁺).

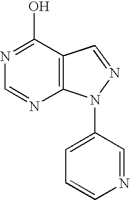

### b. 1-Pyridin-3-yl-1H-pyrazolo[3,4-d]pyrimidin-4-ol

5-amino-1-pyridin-3-yl-1H-pyrazole-4-carbonitrile (0.295 g, 1.59 mmol) was dissolved in 30 mL of formic acid and refluxed for ca. 26 h. The mixture was cooled to RT, concentrated under reduced pressure and diluted with ether. The resulting solid was collected by filtration and washed with ether to give the product as a white solid (0.285 g. 84%).

¹H NMR (DMSO) δ 12.54 (brs, 1H), 9.25 (d,₁H), 8.60 (d, 1H), 8.42 (m, 2H), 8.24 (s, 1H), 7.62 (dd, 1H) ppm; ES-MS m/z 212 (M−H⁻).

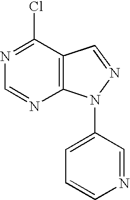

### c. 4-Chloro-1-pyridin-3-yl-1H-pyrazolo[3,4-d]pyrimidine

1-pyridin-3-yl-1H-pyrazolo[3,4-d]pyrimidin-4-ol (0.28 g, 1.3 mmol) was dissolved in phosphorous oxychloride (10 mL) and 2-3 drops of DMF was added. The mixture was heated at reflux for ca. 6.5 h. The mixture was concentrated under reduced pressure and quenched into an ice sodium bicarbonate mixture and extracted with methylene chloride. The organic phase was washed with aqueous sodium bicarbonate and concentrated to give the product as a pale yellow solid (0.155 g, 51%).

¹H NMR (DMSO) δ 9.36 (d, 1H), 9.03 (s, 1H), 8.85 (s, 1H), 8.65 (dd, 1H), 8.54 (m, 1H), 7.68 (dd, 1H) ppm.

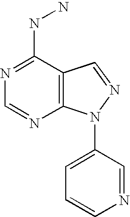

### d. 4-Hydrazino-1-pyridin-3-yl-1H-pyrazolo[3,4-d]pyrimidine

4-chloro-1-pyridin-3-yl-1H-pyrazolo[3,4-d]pyrimidine (0.15 g, 0.65 mmol) was dissolved in ethanol (30 mL) and hydrazine mono-hydrate (0.4 mL, 8.2 mmol) was added. The mixture was heated at 55° C. for ca. 4 h and concentrated under reduced pressure. The resulting solid was triturated with aqueous sodium bicarbonate to give the product as a white solid (0.13 g, 86%).

ES-MS m/z 228 (MH⁺).

### EXAMPLE 1

### Isonicotinaldehyde [1-(6-methoxypyridin-2-yl)-1H-pyrazolo[3,4-d]pyrimidin-4-yl]hydrazone

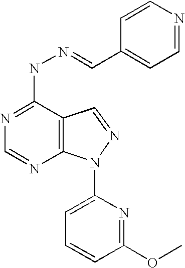

Isonicotinaldehyde (38 mg, 0.35 mmol) and pyrrolidine (1 drop) were added to a stirred solution of 4-hydrazino-1-(6-methoxypyridin-2-yl)-1H-pyrazolo[3,4-d]pyrimidine (Int. Ex. A) (53 mg, 0.20 mmol) in ethanol (3 mL) to give the desired product as a white solid (43 mg, 62%).

¹H NMR (DMSO) δ 12.52 (s, 1H), 8.72 (s, 1H), 8.67 (d, 2H), 8.54, (s, 1H), 8.27 (s, 1H), 7.94 (t, 1H), 7.73 (m, 3H), 6.86 (d, 1H), 3.93 (s, 3H) ppm; ES-MS m/z 347 (MH⁺).

### EXAMPLE 2

### Nicotinaldehyde [1-(6-methoxypyridin-2-yl)-1H-pyrazolo[3,4-d]pyrimidin-4-yl]hydrazone

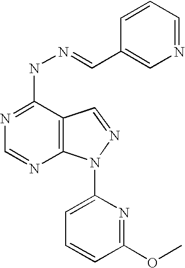

The title compound was prepared according to the general procedure for isonicotinaldehyde [1-(6-methoxypyridin-2-yl)-1H-pyrazolo[3,4-d]pyrimidin-4-yl]hydrazone (Ex. 1) from 4-hydrazino-1-(6-methoxypyridin-2-yl)-1H-pyrazolo[3,4-d]pyrimidine (Int. Ex. A) (61 mg, 0.24 mmol) and nicotinaldehyde (51 mg, 0.47 mmol) to give the product as a white solid (49 mg, 61%).

¹H NMR (DMSO) δ 12.41 (s, 1H), 8.91 (s, 1H), 8.69 (s, 1H), 8.62 (d, 1H), 8.50 (s, 1H), 8.32 (m, 2H), 7.94 (t, 1H), 7.76 (d, 1H), 7.51 (dd, 1H), 6.85 (d, 1H), 3.925 (s, 3H), ppm; ES-MS m/z 347 (MH⁺).

### EXAMPLE 3

### 6-(Dimethylamino)nicotinaldehyde [1-(6-methoxypyridin-2-yl)-1H-pyrazolo[3,4-d]pyrimidin-4-yl]hydrazone

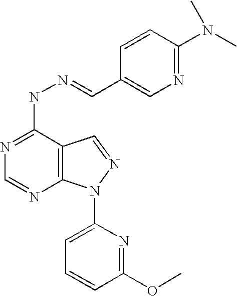

The title compound was prepared according to the general procedure for isonicotinaldehyde [1-(6-methoxypyridin-2-yl)-1H-pyrazolo[3,4-a]pyrimidin-4-yl]hydrazone (Ex. 1) from 4-hydrazino-1-(6-methoxypyridin-2-yl)-1H-pyrazolo[3,4-d]pyrimidine (Int. Ex. A) (53 mg, 0.20 mmol) and 6-(dimethylamino)nicotinaldehyde (54 mg, 0.35 mmol) to give the product as a white solid (15 mg, 19%).

¹H NMR (DMSO) δ 12.07 (s, 1H), 8.64 (s, 1H), 8.42 (s, 1H), 8.33 (s, 1H), 8.17 (s, 1H), 8.07 (d, 1H), 7.93 (t, 1H), 7.76 (d, 1H), 6.84 (d, 1H), 6.76 (d, 1H), 3.93 (s, 3H), 3.09 (s, 6H) ppm; ES-MS m/z 390 (MH⁺).

### EXAMPLE 4

### 2-Chloroisonicotin aldehyde [1-(6-methoxypyridin-2-yl)-1H-pyrazolo[3,4-d]pyrimidin-4-yl]hydrazone

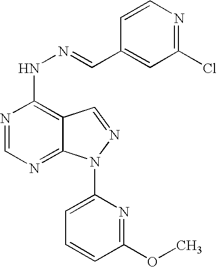

Prepared from 4-hydrazino-1-(6-methoxypyridin-2-yl)-1H-pyrazolo[3,4-d]pyrimidine (Int. Ex. A) and 2-chloroisonicotinaldehyde using the general procedure for isonicotinaldehyde [1-(6-methoxypyridin-2-yl)-1H-pyrazolo[3,4-d]pyrimidin-4-yl]hydrazone (Ex. 1).

¹H NMR (300 MHz, DMSO) δ 12.64 (s, 1H), 8.72 (s, 1H), 8.58 (s, 1H), 8.49 (d, 1H), 8.27 (s, 1H), 8.00-7.71 (m, 4H), 6.87 (d,1H), 3.94 (s, 3H) ppm; ES-MS m/z 381 (MH⁺).

### EXAMPLE 5

### 6-Chloronicotinaldehyde [1-(6-methoxypyridin-2-yl)-1H-pyrazolo[3,4-d]pyrimidin-4-yl]hydrazone

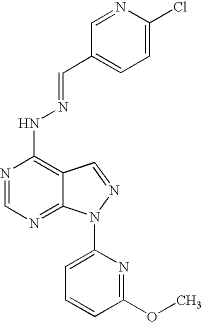

Prepared from 4-hydrazino-1-(6-methoxypyridin-2-yl)-1H-pyrazolo[3,4-d]pyrimidine (Int. Ex. A) and 6-chloronicotinaldehyde using the general procedure for isonicotinaldehyde [1-(6-methoxypyridin-2-yl)-1H-pyrazolo[3,4-d]pyrimidin-4-yl]hydrazone (Ex. 1).

¹H NMR (300 MHz, DMSO) δ 12.47 (s, 1H), 8.75 (s. 1H), 8.71 (s, 1H), 8.52 (s, 1H), 8.40 (d, 1H), 8.33 (s, 1H) 7.95 (t, 1H), 7.78 (d, 1H), 7.60 (d, 1H), 6.87 (d, 1H), 3.94 (s, 3H) ppm; ES-MS m/z 381 (MH⁺).

### EXAMPLE 6

### N-[2-(Dimethylamino)ethyl]-4-((E)-{[1-(6-methoxypyridin-2-yl)-1H-pyrazolo[3,4-d]pyrimidin-4-yl]hydrazono}methyl)benzenesulfonamide

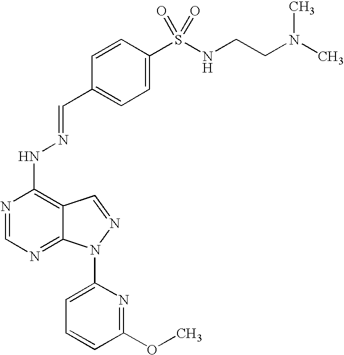

Prepared from 4-hydrazino-1-(6-methoxypyridin-2-yl)-1H-pyrazolo[3,4-d]pyrimidine (Int. Ex. A) and N-[2-(dimethylamino)ethyl]-4-formylbenzenesulfonamide (Int. Ex. F) using the general procedure for isonicotinaldehyde [1-(6-methoxypyridin-2-yl)-1H-pyrazolo[3,4-d]pyrimidin-4-yl]hydrazone (Ex. 1).

¹H NMR (300 MHz, DMSO) δ 12.51 (s, 1H), 8.74 (s, 1H), 8.54 (s, 1H), 8.38 (s, 1H), 8.13-8.03 (m, 3H), 7.94 (t, 3H), 7.79 (d, 1H), 6.87 (d, 1H), 3.94 (s, 3H), 3.12-3.00 (m, 2H), 3.00-2.81 (m, 2H), 2.60 (s, 6H) ppm; ES-MS m/z 496 (MH⁺).

### EXAMPLE 7

### 4-((E)-{[1-(6-methoxypyridin-2-yl)-1H-pyrazolo[3,4-]pyrimidin-4-yl]hydrazono}methyl)-N-(2-morpholin-4-ylethyl)benzenesulfonamide

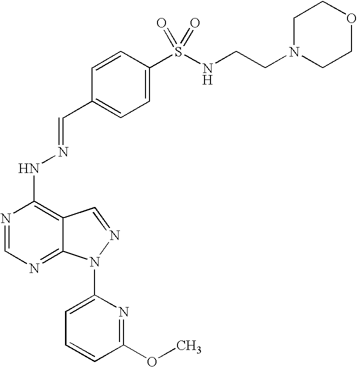

Prepared from 4-hydrazino-1-(6-methoxypyridin-2-yl)-1H-pyrazolo[3,4-d]pyrimidine (Int. Ex. A) and 4-formyl-N-(2-morpholin-4-ylethyl)benzenesulfonamide using the general procedure for isonicotinaldehyde [1-(6-methoxypyridin-2-yl)-1H-pyrazolo[3,4-d]pyrimidin-4-yl]hydrazone (Ex. 1).

1H NMR (300 MHz, DMSO) δ 12.47 (s, 1H), 8.74 (s, 1H), 8.54 (s, 1H), 8.36 (s, 1H), 8.06 (d, 2H), 8.00-7.86 (m, 3H), 7.80 (d, 1H), 7.70 (t, 1H), 6.88 (d, 1H), 3.94 (s, 3H), 3.53-3.47 (m, 4H), 2.92-2.86 (m, 2H), 2.35-2.22 (m, 6H) ppm; ES-MS m/z 538 (MH⁺).

### EXAMPLE 8

### 4-((E)-{[1-(6-methoxypyridin-2-yl)-1H-pyrazolo[3,4-d]pyrimidin-4-yl]hydrazonolmethyl)benzoic acid

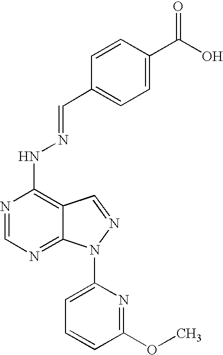

Prepared from 4-hydrazino-1-(6-methoxypyridin-2-yl)-1H-pyrazolo[3,4-d]pyrimidine (Int. Ex. A) and 4-formylbenzoic acid using the general procedure for isonicotinaldehyde [1-(6-methoxypyridin-2-yl)-1H-pyrazolo[3,4-d]pyrimidin-4-yl]hydrazone (Ex. 1).

¹H NMR (300 MHz, DMSO) δ 12.46 (s, 1H), 8.73 (s, 1H), 8.53 (s, 1H), 8.36 (s, 1H), 8.05 (d, 2H), 7.99-7.92 (m, 4H) 7.80 (d, 1H), 6.85 (d. 1H), 3.94 (s, 3H) ppm; ES-MS m/z 388 (MH⁻).

### EXAMPLE 9

### 4-((E)-{[1-(6-methoxypyridin-2-yl)-1H-pyrazolo[3,4-d]pyrimidin-4-yl]hydrazono}methyl)-N-[2-(methylsulfonyl)ethyl]benzamide

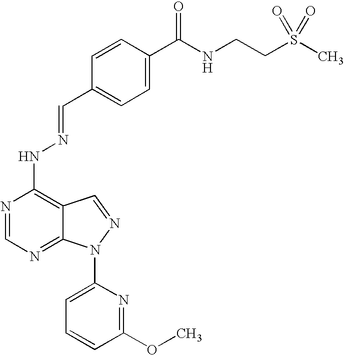

To a solution of 4-((E)-{[1-(6-methoxypyridin-2-yl)-1H-pyrazolo[3,4-d]pyrimidin-4-yl]hydrazono}methyl)benzoic acid (Ex. 8) (47 mg, 0.12 mmol) in DMF (4 ml), was added 2-(methylsulfonyl)ethanamine hydrochloride (29 mg, 0.180 mmol), diethylcyanophosphonate (0.036 ml, 0.240 mmol), and triethylamine (0.05 ml, 0.360 mmol). The solution was stirred at rt for 16 h, then water and diethyl ether were added. The resulting precipitate was collected by filtration to give pure product (48 mg, yield 81%).

¹H NMR (300 MHz, DMSO) δ 12.40 (s, 1H), 8.84 (t, 1H), 8.73 (s, 1H), 8.52 (s, 1H), 8.35 (s, 1H), 8.00-7.92 (m, 5H), 7.79 (d, 1H), 6.87 (d, 1H), 3.94 (s, 3H), 3.71 (q, 2H), 2.45-3.37 (m, 2H), 3.05 (s, 3H) ppm; ES-MS m/z 495 (MH⁺).

### EXAMPLE 10

### N[2-(Dimethylamino)ethyl]-4-((E)-{[1-(6-methoxypyridin-2-yl)-1H-pyrazolo[3,4-d]pyrimidin-4-yl]hydrazono}methyl)benzamide

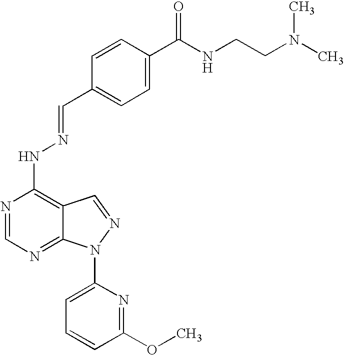

To a solution of 4-((E)-{[1-(6-methoxypyridin-2-yl)-1H-pyrazolo[3,4-d]pyrimidin-4-yl]hydrazono}methyl)benzoic acid (Ex. 8) (47 mg, 0.12 mmol) in DMF (4 mL), was added N,N-dimethylethane-1,2-diamine (29 mg, 0.180 mmol), diethylcyanophosphonate (0.036 mL, 0.240 mmol), and triethylamine (0.05 mL, 0.360 mmol). The solution was stirred at rt for 3 h, then water and diethyl ether were added. The resulting precipitate was collected by filtration to give pure product (33 mg, yield 60%).

¹H NMR (300 MHz, DMSO) δ 12.57-12.16 (s br, 1H), 8.71 (s, 1H), 8.51 (s, 1H), 8.34 (s, 1H), 7.97-7.91 (m, 5H), 7.79 (d, 1H), 6.87 (d, 1H), 3.94 (s, 3H), 3.42-3.34 (m, 2H), 2.49-2.40 (m, 2H), 2.21(s, 6H) ppm; AP-MS m/z 460 (MH⁺).

### EXAMPLE 11

### N-[3-(Dimethylamino)propyl]-4-((E)-{[1-(6-methoxypyridin-2-yl)-1H-pyrazolo[3,4-a]pyrimidin-4-yl]hydrazono}methyl)benzamide

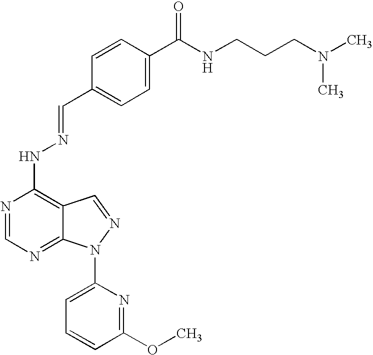

To a solution of 4-((E)-{[1-(6-methoxypyridin-2-yl)-1H-pyrazolo[3,4-d]pyrimidin-4-yl]hydrazono}methyl)benzoic acid (Ex. 8) (47 mg, 0.12 mmol) in DMF (4 mL), was added N,N-dimethylpropane-1,3-diamine (29 mg, 0.180 mmol), diethylcyanophosphonate (0.036 mL, 0.240 mmol), and triethylamine (0.05 mL, 0.360 mmol). The solution was stirred at rt for 3 h, then water and diethyl ether were added. The resulting percipitate was collected by filtration to give pure product (18 mg, yield 32%).

¹H NMR (300 MHz, DMSO) δ 12.56-12.06 (s br,₁H), 8.72 (s, 1H), 8.63 (t, 1H), 8.52 (s, 1H), 8.35 (s, 1H), 8.00-7.91 (m, 5H), 7.80 (d, 1H), 6.87 (d, 1H), 3.94 (s, 3H), 3.42-3.34 (m, 2H), 2.29 (t, 2H), 2.15 (s, 6H) 1.76-1.60 (m, 2H) ppm; AP-MS m/z 475 (MH⁺).

### EXAMPLE 12

### 4-((E)-{[1-(6-methoxypyridin-2-yl)-1H-pyrazolo[3,4-d]pyrimidin-4-yl]hydrazono}methyl)-N-(3-pyrrolidin-1-ylpropyl)benzamide

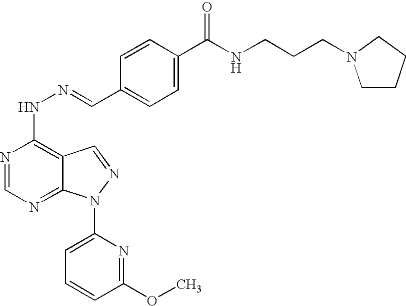

To a solution of 4-((E)-{[1-(6-methoxypyridin-2-yl)-1H-pyrazolo[3,4-d]pyrimidin-4-yl]hydrazono}methyl)benzoic acid (Ex. 8) (50 mg, 0.13 mmol) in DMF (2 ml), was added 3-pyrrolidin-1-ylpropan-1-amine (0.064 ml, 0.51 mmol), diethylcyanophosphonate (0.059 ml, 0.51 mmol), and triethylamine (0.071 ml, 0.51 mmol). The solution was stirred at rt for 1.5 h, then 1/2 sat. NaCl was added. The resulting precipitate was collected by filtration to give product as a brown solid (38 mg, yield 59%).

¹H NMR (300 MHz, DMSO) δ 12.38 (s, 1H), 8.73-7.68 (m, 2H), 8.51 (s, 1H), 8.35 (s, 1H), 7.98-7.89 (m, 5H), 7.79 (d, 1H), 6.87 (d, 1H), 3.93 (s, 3H), 3.40-3.28 (m, 2.55-2.39 (m, 6H), 1.75-1.63 (m, 6H) ppm; ES-MS m/z 500 (MH⁺).

### EXAMPLE 13

### Nicotinaldehyde (1-pyridin-2-yl-1H-pyrazolo[3,4-d]pyrimidin-4-yl)hydrazone

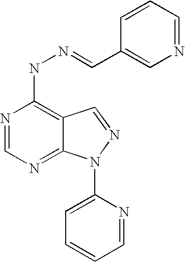

The title compound was prepared according to the general procedure for isonicotinaldehyde [1-(6-methoxypyridin-2-yl)-1H-pyrazolo[3,4-d]pyrimidin-4-yl]hydrazone (Ex. 1) from 4-hydrazino-1-pyridin-2-yl-1H-pyrazolo[3,4-d]pyrimidine (Int. Ex. B) (70 mg, 0.31 mmol) and nicotinaldehyde (50 mg, 0.467 mmol) to give the product as a white solid (35 mg, 36%).

¹H NMR (DMSO) δ 12.43 (s, 1H), 8.93 (s, 1H), 8.69 (s, 1H), 8.62 (d, 2H), 8.51 (s, 1H), 8.35 (s, 1H), 8.32 (d, 1H), 8.12 (d, 1H), 8.07 (dt, 1H), 7.53 (dd, 1H), 7.47 (t, 1H) ppm; ES-MS m/z 317 (MH⁺).

### EXAMPLE 14

### Isonicotinaldehyde (1-pyridin-2-yl-1H-pyrazolo[3,4-d]pyrimidin-4-yl)hydrazone

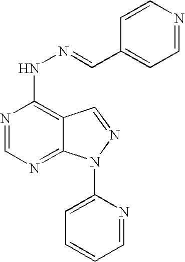

Prepared from 4-hydrazino-1-pyridin-2-yl-1H-pyrazolo[3,4-d]pyrimidine (Int. Ex. B) and isonicotinaldehyde using the general procedure for isonicotinaldehyde [1-(6-methoxypyridin-2-yl)-1H-pyrazolo[3,4-d]pyrimidin-4-yl]hydrazone (Ex. 1).

¹H NMR (400 MHz, DMSO) δ 12.53 (s, 1H), 8.74 (d, 2H), 8.70-8.67 (m, 2H), 8.67-8.61 (m, 1H), 8.30 (s, 1H), 8.15-8.00 (m, 2H), 7.83-7.78 (m, 3H), 7.50-7.45 (m, 1H) ppm; ES-MS m/z 317 (MH⁺).

### EXAMPLE 15

### tert-Butyl 4-{(E)-[(1-pyridin-2-yl-1H-pyrazolo[3,4-d]pyrimidin-4-yl)hydrazono]methyl}piperidine-1-carboxylate

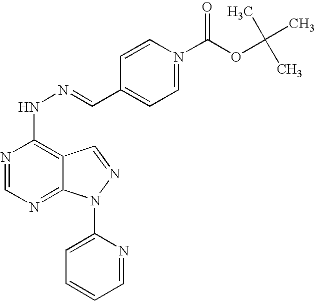

Prepared from 4-hydrazino-1-pyridin-2-yl-1H-pyrazolo[3,4-d]pyrimidine (Int. Ex. B) and tert-butyl 4-formylpiperidine-1-carboxylate using the general procedure for isonicotinaldehyde [1-(6-methoxypyridin-2-yl)-1H-pyrazolo[3,4-d]pyrimidin-4-yl]hydrazone (Ex. 1).

¹H NMR (300 MHz, DMSO) δ 11.87 (s, 1H), 8.60 (s, 1H), 8.43 (d, 2H), 8.29 (s, 1H), 8.09 (d, 1H), 8.04 (t, 1H), 7.58 (s, 1H), 7.43 (t, 1H), 4.03-3.96 (m, 2H), 2.90-2.80 (m, 2H), 2.65-2.58 (m, 1H), 1.94-1.86 (m, 2H), 1.39 (s, 9H), 1.28-1.18 (m, 2H) ppm.

### EXAMPLE 16

### tert-Butyl 5-{(E)-[(1-pyridin-2-yl-1H-pyrazolo[3,4-d]pyrimidin-4-yl)hydrazono]methyl}pyridin-2-ylcarbamate

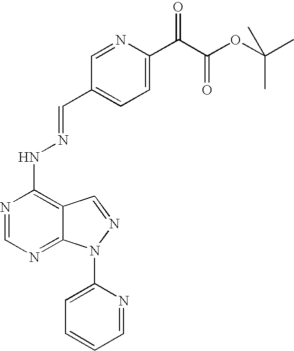

Prepared from 4-hydrazino-1-pyridin-2-yl-1H-pyrazolo[3,4-d]pyrimidine (Int. Ex. B) and tert-butyl 5-formylpyridin-2-ylcarbamate using the general procedure for isonicotinaldehyde [1-(6-methoxypyridin-2-yl)-1H-pyrazolo[3,4-d]pyrimidin-4-yl]hydrazone (Ex. 1).

¹H NMR (300 MHz, DMSO) δ 12.29 (s, 1H), 10.07 (s, 1H), 8.67 (s, 1H), 8.62 (d, 1H), 8.57 (s, 1H), 8.47 (s, 1H), 8.34-8.23 (m, 2H), 8.16-8.09 (m, 1H), 8.08-8.01 (m, 1H), 7.95 (d, 1H), 7.45 (t, 1H), 1.48 (s, 9H) ppm; AP-MS m/z 430 (MH⁻).

### EXAMPLE 17

### 6-Aminonicotinaldehyde (1-pyridin-2-yl-1H-pyrazolo[3,4-d]pyrimidin-4-yl)hydrazone trifluoroacetate

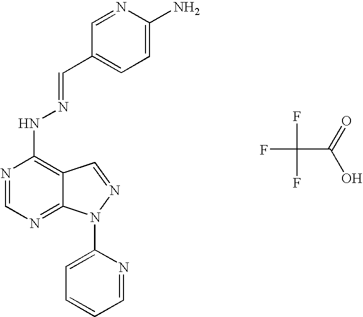

A solution of tert-butyl 5-{(E)-[(1-pyridin-2-yl-1H-pyrazolo[3,4-d]pyrimidin-4-yl)hydrazono]methyl}pyridin-2-ylcarbamate (Ex. 16) (44 mg, 0.102 mmol), dichloromethane (5 ml), and TFA (1 ml) was stirred at rt for 8 days. The mixture was concentrated, dichloromethane was added resulting solid was filtered and collected to give 6-aminonicotinaldehyde (1-pyridin-2-yl-1H-pyrazolo[3,4-d]pyrimidin-4-yl)hydrazone trifluoroacetate as pure product (38 mg, 89% yield).

¹H NMR (300 MHz, DMSO) d 12.83-12.16 (s br, 1H), 8.71 (s, 1H), 8.62 (d, 1H), 8.54 (d, 1H), 8.50 (s, 1H), 8.45-8.12 (m, 1H), 8.33 (s, 1H), 8.40-8.30 (m, 2H), 8.14-8.02 (m, 2H), 7.52-7.44 (m, 1H), 7.11 (d, 1H) ppm; AP-MS m/z 332 (MH⁺).

### EXAMPLE 18

### 6-(Dimethylamino)nicotinaldehyde (1-pyridin-2-yl-1H-pyrazolo[3,4-d]pyrimidin-4-yl)hydrazone

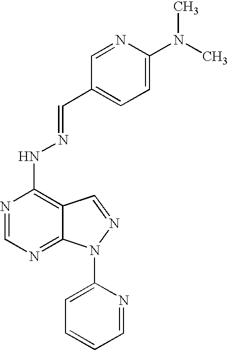

Prepared from 4-hydrazino-1-pyridin-2-yl-1H-pyrazolo[3,4-d]pyrimidine (Int. Ex. B) and 6-(dimethylamino)nicotinaldehyde using the general procedure for isonicotinaldehyde [1-(6-methoxypyridin-2-yl)-1H-pyrazolo[3,4-d]pyrimidin-4-yl]hydrazone (Ex. 1).

¹H NMR (300 MHz, DMSO) δ 12.09 (s, 1H), 8.68-8.58 (m, 2H), 8.43 (s, 1H), 8.35 (s, 1H), 8.19 (s, 1H), 8.16-8.11 (m, 1H), 8.09-8.04 (m, 2H), 7.51-7.41 (m, 1H), 6.79 (d, 1H), 3.11 (s, 6H) ppm; ES-MS m/z 360 (MH⁺).

### EXAMPLE 19

### N-[2-(Dimethylamino)ethyl]-4-{(E)-[(1-pyridin-2-yl-1H-pyrazolo[3,4-d]pyrimidin-4-yl)hydrazono]methyl}benzenesulfonamide

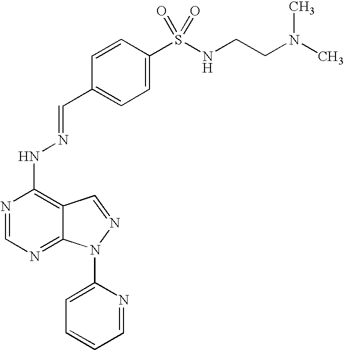

Prepared from 4-hydrazino-1-pyridin-2-yl-1H-pyrazolo[3,4-d]pyrimidine (Int. Ex. B) and N-[2-(dimethylamino)ethyl]-4-formylbenzenesulfonamide (Int. Ex. F) using the general procedure isonicotinaldehyde [1-(6-methoxypyridin-2-yl)-1H-pyrazolo[3,4-d]pyrimidin-4-yl]hydrazone (Ex. 1).

¹H NMR (300 MHz, DMSO) δ 12.45 (s, 1H), 8.71(s, 1H), 8.63 (d, 1H), 8.53 (s, 1H), 8.39 (s, 1H), 8.14-8.02 (m, 4H), 7.98-7.90 (m, 3H), 7.51-7.41 (m, ₁H), 3.32 (s, 3.11-2.96 (m, 2H), 2.94-2.75 (m, 2H) ppm; ES-MS m/z 466 (MH⁺).

### EXAMPLE 20

### 4-{(E)-[(1-pyridin-2-yl-1H-pyrazolo[3,4-d]pyrimidin-4-yl)hydrazono]methyl}benzoic acid

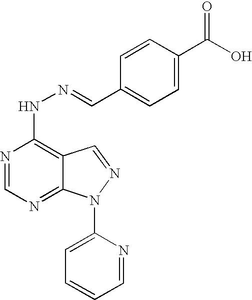

Prepared from 4-hydrazino-1-pyridin-2-yl-1H-pyrazolo[3,4-d]pyrimidine (Int. Ex. B) and 4-formylbenzoic acid using the general procedure for isonicotinaldehyde [1-(6-methoxypyridin-2-yl)-1H-pyrazolo[3,4-d]pyrimidin-4-yl]hydrazone (Ex. 1).

¹H NMR (400 MHz, DMSO) δ 12.48 (s, 1H), 8.70 (s, 1H), 8.64 (d, 1H), 8.52 (s, 1H), 8.38 (s, 1H), 8.16-8.00 (m, 4H), 7.96 (d, 2H), 7.50-7.44 (m, 1H) ppm; AP-MS m/z 360 (MH⁺).

### EXAMPLE 21

### N-[2-(Dimethylamino)ethyl]-4-{(E)-[(1-pyridin-2-yl-1H-pyrazolo[3,4-d]pyrimidin-4-yl)hydrazono]methyl}benzamide hydrochloride

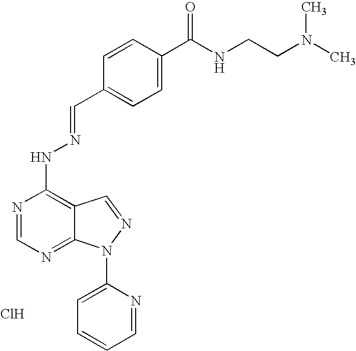

To a solution of 4-{(E)-[(1-pyridin-2-yl-1H-pyrazolo[3,4-d]pyrimidin-4-yl)hydrazono]methyl}benzoic acid (Ex. 20) (43 mg, 0.12 mmol) in DMF (4 mL), was added N,N-dimethylethane-1,2-diamine (0.02 mL, 0.180 mmol), diethylcyanophosphonate (0.036 mL, 0.240 mmol), and triethylamine (0.05 mL, 0.360 mmol). The solution was stirred at rt for 1 h, then partitioned between water and diethyl ether. The aqueous layer was made acidic with 1N HCl, then concentrated. The resulting solid was washed with ethanol and diethyl ether, and collected by filtration to give pure product (45 mg, yield 81%).

¹H NMR (300 MHz, DMSO) δ 10.08 (s, 1H), 8.99-8.92 (m, 1H), 8.72 (s, 1H), 8.63 (d, 1H), 8.55 (s, 1H), 8.41 (s, 1H), 8.21-7.90 (m, 6H), 7.50-7.42 (m, 1H), 3.71-3.62 (m, 2H), 3.32-3.25 (m, 2H), 2.84 (s, 6H) ppm; ES-MS m/z 430 (MH⁺).

### EXAMPLE 22

### N-[2-(Methylsulfonyl)ethyl]-4-{(E)-[(1-pyridin-2-yl-1H-pyrazolo[3,4-d]pyrimidin-4-yl)hydrazono]methyl}benzamide

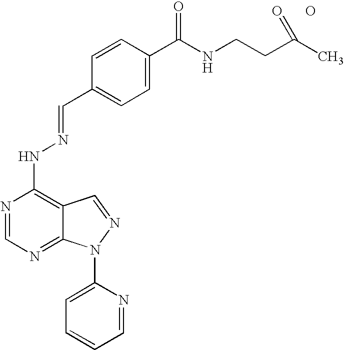

To a solution of 4-{(E)-[(1-pyridin-2-yl-1H-pyrazolo[3,4-d]pyrimidin-4-yl)hydrazono]methyl}benzoic acid (Ex. 20) (43 mg, 0.12 mmol) in DMF (4 ml), was added 2-(methylsulfonyl)ethanamine hydrochloride (29 mg, 0.180 mmol), diethylcyanophosphonate (0.036 ml, 0.240 mmol), and triethylamine (0.05 ml, 0.360 mmol). The solution was stirred at rt for 16 h, then water and diethyl ether were added. The resulting precipitate was collected by filtration to give pure product (38 mg, yield 68%).

¹H NMR (300 MHz, DMSO) δ 12.40 (s, 1H), 8.83 (t, 1H), 8.70 (s, 1H), 8.64 (s, 1H), 8.52 (s, 1H), 8.36(s,1H), 8.18-8.02 (m, 2H), 7.94 (s, 4H), 7.51-7.43 (m, 1H), 3.71 (q, 2H), 3.38 (q, 2H), 3.05 (s, 3H) ppm; ES-MS m/z 465 (MH⁺).

### EXAMPLE 23

### Nicotinaldehyde [1-(1,3-thiazol-2-yl)-1H-pyrazolo[3,4-d]pyrimidin-4-yl]hydrazone

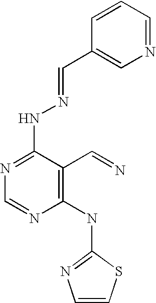

4-Hydrazino-1-(1,3-thiazol-2-yl)-1H-pyrazolo[3,4-d]pyrimidine (Int. Ex. C) (0.051 g, 0.22 mmol) was treated with nicotinaldehyde (0.070 g, 0.66 mmol) in absolute ethanol as described for isonicotinaldehyde [1-(6-methoxypyridin-2-yl)-1H-pyrazolo[3,4-d]pyrimidin-4-yl]hydrazone (Ex. 1) to give 49.7 mg (70%) of product as an off-white solid.

¹H NMR (DMSO) δ12.50 (s, 1H), 8.92 (s, 1H), 8.73 (s, 1H), 8.61 (d, 2H), 8.35 (m, 2H), 7.70 (dd, 2H), 7.52 (m, 1H) ppm; ES-MS m/z 323 (MH⁺)

### EXAMPLE 24

### Isonicotinaldehyde [1-(1,3-thiazol-2-yl)-1H-pyrazolo[3,4-d]pyrimidin-4-yl]hydrazone

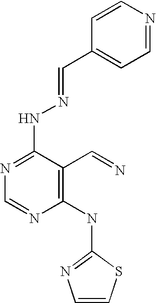

4-Hydrazino-1-(1,3-thiazol-2-yl)-1H-pyrazolo[3,4-d]pyrimidine (Int. Ex. C) (0.052 g, 0.223 mmol) was treated with isonicotinaldehyde as described for isonicotinaldehyde [1-(6-methoxypyridin-2-yl)-1H-pyrazolo[3,4-d]pyrimidin-4-yl]hydrazone (Ex. 1) to give 47.8 mg (66%) of product as an off-white solid.

¹H NMR (DMSO) δ 12.65 (br s, 1H), 8.75 (s, 1H), 8.67 (d, 2H), 8.62 (s, 1H), 8.29 (s, 1H), 7.80 (d, 2H), 7.75 (d, 1H), 7.70 (d, 1H) ppm; ES-MS m/z 323 (MH⁺)

### EXAMPLE 25

### N-[2-(Dimethylamino)ethyl]-4-((E)-{[1-(1,3-thiazol-2-yl)-1H-pyrazolo[3,4-a]pyrimidin-4-yl]hydrazonol}methyl)benzenesulfonamide

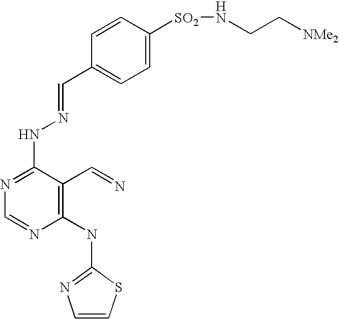

4-Hydrazino-1-(1,3-thiazol-2-yl)-1H-pyrazolo[3,4-d]pyrimidine (Int. Ex. C) (0.054 g, 0.232 mmol) was treated with N-[2-(dimethylamino)ethyl]-4-formylbenzenesulfonamide hydrochloride (Int. Ex. F) (0.125 g, 0.427 mmol) as described for isonicotinaldehyde [1-(6-methoxypyridin-2-yl)-1H-pyrazolo[3,4-d]pyrimidin-4-yl]hydrazone (Ex. 1) to give 0.0914 g (84%) of product as an off-white solid.

¹H NMR (DMSO) δ 9.70 (br s, 1H), 8.75 (s,1H), 8.61 (s, 1H), 8.38 (s, 1H), 8.07 (d, 2H), 8.00 (br s, 1H), 7.91 (d, 2H), 7.75 (d, 1H), 7.68 (d, 1H), 3.03 (s, 6H), 3.05 (m, 2H), 2.60 (m, 2H) ppm; ES-MS m/z 472 (MH⁺)

### EXAMPLE 26

### Isonicotinaldehyde (1-pyridin-4-yl-1H-pyrazolo[3,4-d]pyrimidin-4-yl)hydrazone

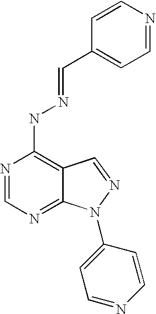

To a suspension of 4-hydrazino-1-pyridin-4-yl-1H-pyrazolo[3,4-d]pyrimidine (Int. Ex. D) (100 mg, 0.44 mmol) in ethanol (7 ml) was added 4-pyridylcarboxaldehyde (0.13 ml,1.32 mmol). The mixture was refluxed for 2 hours. The solid was filtered, washed with ethanol (3×) and dried to give product as a solid (92 mg, 66%).

¹H NMR (400 MHz CD₃OD with DCl) δ 9.16 (d, 2H), 9.15 (s, 1H), 9.09 (d, 2H), 9.01 (d, 2H), 8.99 (s, 1H), 9.0(s. 1H), 8.81 (d, 2H) ppm; ES-MS m/z 317 (MH⁺).

### EXAMPLE 27

### Isonicotinaldehyde (1-pyridin-4-yl-1H-pyrazolo[3,4-d]pyrimidin-4-yl)hydrazone triplehydrochloride

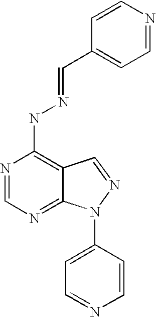

Isonicotinaldehyde (1-pyridin-4-yl-1H-pyrazolo[3,4-d]pyrimidin-4-yl)hydrazone (Ex. 26) (17 mg, 0.054 mmol) was dissolved in 1N hydrochloric acid (0.16 ml, 0.16 mmol). The solution was then lyophilized to give product as a solid quantitatively.

¹H NMR (300 MHz, CD3OD), δ 9.28 (d, 2H), 9.04 (br, 1H), 8.98 (d, 2H), 8.94 (d, 2H), 8.83 (s, 1H), 8.50 (d, 2H), 8.47 (s, 1H) ppm; ES-MS m/z 317 (MH⁺).

### EXAMPLE 28

### Isonicotinaldehyde [1-(1H-benzimidazol-4-yl)-1H-pyrazolo[3,4-d]pyrimidin-4-yl]hydrazone

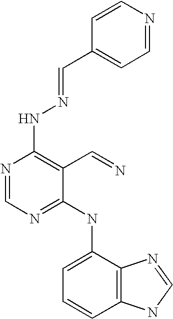

1-(1H-Benzimidazol-4-yl)-4-hydrazino-1H-pyrazolo[3,4-d]pyrimidine (Int. Ex. E) (0.025 g, 0.094 mmol) was treated with isonicotinaldehyde as described for isonicotinaldehyde [1-(6-methyloxypyridin-2-yl)-1H-pyrazolo[3,4-d]pyrimidin-4-yl]hydrazone (Ex. 1) to give 0.020 g (60%) of product as a light yellow solid.

¹H NMR (DMSO) δ 12.60 (br s, 1H), 8.74 (s, 1H), 8.69 (d, 2H), 8.49 (s, 1H), 8.30 (s, 2H), 7.88 (br s, 1H), 7.79 (d, 2H), 7.72 (d, 1H), 7.38 (t, 1H) ppm; ES-MS m/z 355 (MH⁺).

### EXAMPLE 29

### 4-Fluorobenzaldehyde [1-(1H-benzimidazol-4-yl)-1H-pyrazolo[3,4-d]pyrimidin-4-yl]hydrazone

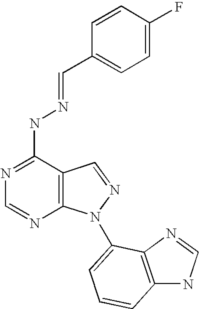

1-(1H-Benzimidazol-4-yl)-4-hydrazino-1H-pyrazolo[3,4-d]pyrimidine (Int. Ex. E) (0.050 g, 0.188 mmol) was treated with 4-fluorobenzaldehyde as described for isonicotinaldehyde [1-(6-methyloxypyridin-2-yl)-1H-pyrazolo[3,4-d]pyrimidin-4-yl]hydrazone (Ex. 1) to give 0.026 g (34%) of product as an off-white solid.

1H NMR (300 MHz, DMSO-d6) 12.5 (br s, 1H), 8.7 (s, 1H), 8.3 (m, 3H), 7.8-8.0 (m, 3H), 7.5 (d, 1H), 7.3 (m, 4H). ES-MS m/z 373 (MH⁺).

### EXAMPLE 30

### 4-(Methylsulfonyl)benzaldehyde [1-(1H-benzimidazol-4-yl)-1H-pyrazolo[3,4-d]pyrimidin-4-yl]hydrazone

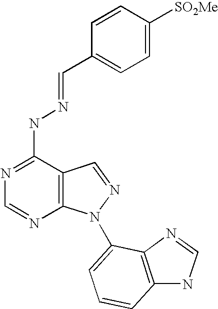

1-(1H-Benzimidazol-4yl)-4-hydrazino-1H-pyrazolo[3,4-d]pyrimidine (Int. Ex. E) (0.050 g, 0.188 mmol) was treated with 4-methylsulfonylbenzaldehyde as described for isonicotinaldehyde [1-(6-methyloxypyridin-2-yl)-1H-pyrazolo[3,4-d]pyrimidin-4-yl]hydrazone (Ex. 1) to give 0.027 g (33%) of product as an off-white solid.

1H NMR (400 MHz, DMSO) 12.50 (br s, 1H), 8.7 (m, 1H), 8.3-8.6 (m,2H), 8.25 (s, 1H), 8.0-8.2 (m, 5H), 7.7 (m, 1H), 7.40 (m, 1H), 3.3 (s, 3H). ES-MS m/z 433 (MH⁺).

### EXAMPLE 31

### 4-((E)-{[1-(1H-benzimidazol-4-yl)-1H-pyrazolo[3,4-d]pyrimidin-4-yl]hydrazono}methyl)benzoic acid

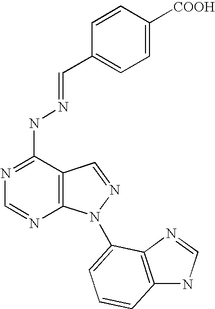

1-(1H-Benzimidazol-4-yl)-4hydrazino-1H-pyrazolo[3,4-d]pyrimidine (Int. Ex. E) (0.050 g, 0.188 mmol) was treated with 4-carboxybenzaldehyde as described for isonicotinaldehyde [1-(6-methyloxypyridin-2-yl)-1H-pyrazolo[3,4-d]pyrimidin-4-yl]hydrazone (Ex. 1) to give 0.087 g (81%) of product as an off-white solid.

1H NMR (400 MHz, DMSO) 13.0 (br s, 1H), 12.50 (br s, 1H), 8.8 (m, 1H), 8.2-8.6 (m, 3H), 7.8-8.2 (m, 5H), 7.8 (m, 1H), 7.40 (m, 1H). ES-MS m/z 399 (MH⁺).

### EXAMPLE 32

### 4-((E)-{[1-(1H-benzimidazol-4-yl)-1H-pyrazolo[3,4-d]pyrimidin-4-yl]hydrazono}methyl)-N-[3-(dimethylamino)propyl]benzamide

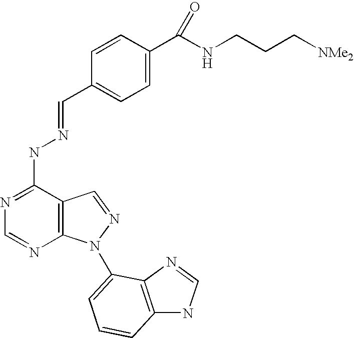

A mixture of 4-((E)-{[1-(1H-benzimidazol-4-yl)-1H-pyrazolo[3,4-d]pyrimidin-4-yl]hydrazono}methyl)benzoic acid (0.060 g, 0.150 mmol), N,N-dimethylpropane-1,3-diamine (25 μL, 0.225 mmol), diethyl cyanophosphonate (45 μL, 0.300 mmol), triethylamine (63 μl, 0.45 mmol) in 3 mL of dimethylformamide was stirred at room temperature under nitrogen for 1.5 hours. The reaction mixture was concentrated in vacuo and water was added. The precipitated solid was collected by filtration, washed with water and methanol and dried under vacuum to give 8.6 mg (12%) of a yellow solid.

1H NMR (400 MHz, DMSO) 13.0 (br s, 1H), 12.40 (br m, 2H), 8.8 (s, 1H), 8.5 (m, 2H), 8.3 (m, 1H), 8.25 (m, 1H), 8.1 (m, 1H), 7.9 (m, 4H), 7.60 (m, 1H), 7.40(m, 1H), 3.3 (m,2H), 2.4 (m,2H), 2.2 (s, 6H). ES-MS m/z 469 (MH⁺).

### EXAMPLE 33

### 4-Fluorobenzaldehyde (1-pyridin-3-yl-1H-pyrazolo[3,4-d]pyrimidin-4-yl)hydrazone

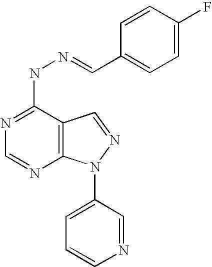

The title compound was prepared according to the general procedure for isonicotinaldehyde [1-(6-methoxypyridin-2-yl)-1H-pyrazolo[3,4-d]pyrimidin-4-yl]hydrazone (Ex. 1) from 4-Hydrazino-1-pyridin-3-yl-1H-pyrazolo[3,4-d]pyrimidine (Intermediates Example G) (65 mg, 0.29 mmol) and 4-fluorobenzaldehyde (71 mg, 0.57 mmol) to give the product as a white solid (76 mg, 61%).

¹H NMR (DMSO) δ 12.33 (s, 1H), 9.43 (d, 1H), 8.70 (s, 1H), 8.58 (m, 2H), 8.52 (s, 1H), 8.31 (s, 1H), 7.91 (dd, 2H), 7.63 (dd, 1H), 7.33 (t, 2H) ppm; ES-MS m/z 334 (MH⁺).

Biological Data

GSK3

The compounds of the present invention elicit important and measurable pharmacological responses. In evaluating those responses, the present invention also demonstrated unexpected advantageous biological and pharmacological properties. In short, the present invention provides unexpected superior performance characteristics not heretofore appreciated.

The protocol used to demonstrate the pharmacological response of the present invention is based on the ability of the kinase to phosphorylate a biotinylated peptide, the sequence of which is derived from the phosphorylation site of glycogen synthase and its sequence is: Biotin-Ahx-AAAKRREILSRRPS(PO₃)YR-amide. The phosphorylated biotinylated peptide is then captured onto streptavidin coated scintillation proximity assay (SPA) beads from Amersham Technology, where the signal from the ³³P is amplified via the scintillant contained in the beads.

GSK-3β is commercially available or may be cloned and expressed in E coli using standard techniques to produce soluble, active protein. The production of active protein involves purification in two steps using Metal Chelate and Ion Exchange Chromatography. Protein eluting from Ion Exchange provides >90% pure product that may then be concentrated for use in high throughput screening.

The kinase was assayed at a concentration of 20 nM final in 100 mM HEPES, pH 7.2 containing 10 mM magnesium chloride, 0.1 mg/mL bovine serum albumin, 1 mM dithiothreitol, 0.3 mg/mL heparin, 2.8 uM peptide substrate, 2.5 uM ATP, and 0.2 uCi/well γ³³P]-ATP. After 40 minutes incubation at room temperature, the reaction was stopped by addition of 100 mM EDTA and 1 mM solution in 100 mM HEPES, pH7.2 followed by an additional solution of diluted Streptavidin coated SPA beads in PBS, pH 7.2 to give a final concentration of 0.25 mg of beads per assay well in a 96-well microtiter plate.

10 mM stock solutions of the compounds of the invention in 100% DMSO are generated as a first step in the screening process. The second step involves the creation of dose response plates where these compounds are diluted 10-fold in 100% DMSO to 1 mM concentrations and subsequently serially diluted 3-fold in 100% DMSO across the plate by automated liquid handling such that the final top concentration of inhibitor is 0.033 mM in the 30 uL kinase assay. The third step involves the creation of the assay plates. This is achieved by transferring 1 uL of the compounds to assay plates by automated liquid handling. The fourth step is to perform the assay as described and count the resulting plates in the Packard TopCount NXT microplate scintillation and luminescence counter.

The final step is data acquisition and analysis where IC₅₀ values are generated for each compound by normalizing curve data to the equation 100*(U1−C2)/(C1−C2) (where U1 is the cpm value, C2 is the background, and C1 is the maximum number of counts), then fitting the normalized data to the equation y=Vmax*(1−(x/(K+x))). The IC₅₀ values were converted to pIC₅₀ values, i.e., −log IC₅₀ in Molar concentration. The data is expressed below in Table 1.

TABLE 1

| Example | GSK pIC50 |
|---|---|
| Example 1 | +++ |
| Example 2 | +++ |
| Example 3 | ++ |
| Example 4 | ++ |
| Example 5 | +++ |
| Example 6 | +++ |
| Example 7 | +++ |
| Example 8 | ++ |
| Example 9 | +++ |
| Example 10 | +++ |
| Example 11 | +++ |
| Example 12 | +++ |
| Example 13 | ++ |
| Example 14 | + |
| Example 15 | + |
| Example 17 | + |
| Example 18 | + |
| Example 19 | ++ |
| Example 20 | + |
| Example 21 | ++ |
| Example 22 | ++ |
| Example 23 | ++ |
| Example 24 | +++ |
| Example 25 | +++ |
| Example 26 | +++ |
| Example 27 | +++ |
| Example 28 | +++ |
| Example 29 | +++ |
| Example 30 | +++ |
| Example 31 | +++ |
| Example 32 | +++ |
| Example 33 | +++ |

+ = pIC₅₀ of 5.0-6.0; ++ = pIC₅₀ of 6.0-7.0; +++ = pIC₅₀ of >7.0.

Test compounds are employed in free or salt form.

All research complied with the principles of laboratory animal care (NIH publication No. 85-23, revised 1985) and GlaxoSmithKline policy on animal use.

Although specific embodiments of the present invention have been illustrated and described in detail, the invention is not limited thereto. The above detailed description of preferred embodiments is provided for example only and should not be construed as constituting any limitation of the invention. Modifications will be obvious to those skilled in the art, and all modifications that do not depart from the spirit of the invention are intended to be included within the scope of the appended claims.

What is claimed is:

1. A compound of Formula (I) 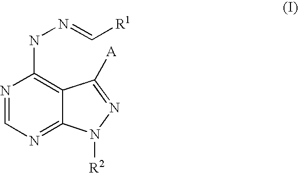
including salts, solvates, and pharmaceutically acceptable derivatives thereof,
wherein A is H, alkyl, or aryl;
R¹ is D¹, D², D³, D⁴, or D⁵,
wherein D¹ is 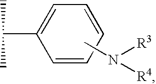
and R³ and R⁴ are each independently H, alkyl, alkylsulfonyl, or —C(O)—(CH₂)ₓ—R⁵,
where R⁵ is alkyl, acyl, alkoxy, —(O)—(CH₂)ₓ—(O)-alkyl, or —NR⁶R⁷,
where R⁶ and R⁷ are each independently H or alkyl, or
R⁶ and R⁷ combine to form a 5- or 6-membered ring, optionally containing one or more additional heteroatoms, optionally containing one or more degrees of unsaturation, and optionally substituted one or more times with alkyl, hydroxy, carboxy, acyl, alkoxy, or halogen,
or R³ and R⁴ combine to form a 5- or 6-membered ring, optionally containing one or more additional heteroatoms, optionally containing one or more degrees of unsaturation, and optionally substituted one or more times with alkyl, hydroxy, carboxy, alkoxy, acyl, or halogen;
wherein D² is 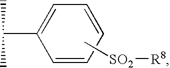
and R⁸ is alkyl, or —NR⁹R¹⁰,
where R⁹ and R¹⁰ are each independently selected from H, alkyl, or —(CH₂)ₓ—NR⁶R⁷,
where R⁶ and R⁷ are each independently H or alkyl,
or R⁶ and R⁷ combine to form a 5- or 6-membered ring, optionally containing one or more additional heteroatoms, optionally containing one or more degrees of unsaturation, and optionally substituted one or more times with alkyl, hydroxy, carboxy, acyl, alkoxy, or halogen;
wherein D³ is 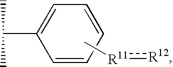
and
the dashed line represents an optional double bond;
when R¹¹ is —(CH₂)ₓ, the optional dashed double bond does not exist, and R¹² is alkylsulfonyl or —NR¹³R¹⁴,
where R¹³ and R¹⁴ are each independently selected from H, alkyl, —(CH₂)ₓ—R¹⁷, where R¹⁷ is alkoxy or —NR¹⁵R¹⁶,
where R¹⁵ and R¹⁶ are each independently H or alkyl,
or R¹³ and R¹⁴ combine to form a 5- or 6-membered ring, optionally containing one or more additional heteroatoms, optionally containing one or more degrees of unsaturation, and optionally substituted one or more times with alkyl or —(CH₂)ₓ—OH;
when R¹¹ is —(CH)—, the optional dashed double bond exists, and R¹² is —(CH)—C(O)—OH;
wherein D⁴ is 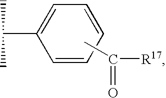
and R¹⁷ is hydroxy, alkoxy, or —NR¹⁸ R¹⁹,
where R¹⁸ and R¹⁹ are each independently selected from H, alkyl, —(CH₂)ₓ—R²⁰,
where R²⁰ is alkylsulfonyl, hydroxy, aryl said aryl optionally substituted with hydroxy or alkoxy, heteroaryl, or —NR²¹R²²,
where R²¹ and R²² are each independently selected from H, acyl, alkyl,
or R²¹ and R²² combine to form a 5- or 6-membered ring, optionally containing one or more additional heteroatoms, optionally containing one or more degrees of unsaturation, and optionally substituted with alkyl or —(CH₂)ₓ—OH;
or R¹⁸ and R¹⁹ combine to form a 5- or 6-membered ring, optionally containing one or more additional heteroatoms, optionally containing one or more degrees of unsaturation, and optionally substituted with —(CH₂)ₓ—R²³,
where R²³ is alkoxy, hydroxy, —C(O)—R²⁴, where R²⁴ is a 5- or 6-membered ring optionally containing one or more heteroatoms and optionally containing one or more degrees of unsaturation, or —NR²⁵R²⁶, where R²⁵ and R²⁶ are each independently H or alkyl;
wherein D⁵ is
a 5- or 6-membered ring, optionally containing one or more heteroatoms, optionally containing one or more degrees of unsaturation, optionally fused with an additional 5- or 6-membered ring that optionally contains one or more heteroatoms and optionally contains one or more degrees of unsaturation,
wherein the ring or fused ring system may be optionally substituted one or more times with halogen, alkyl, haloalkyl, alkylsulfonyl, alkylthio, hydroxy, alkoxy, oxo, sulfonyl, sulfate ion, nitro, cyano, carboxy, alkoxycarbonyl, aryl where said aryl may be optionally substituted with sulfamoyl, heteroaryl where said heteroaryl may be optionally substituted with alkyl, or —NR²⁷R²⁸,
where R²⁷ and R²⁸ are each independently H, alkyl, acyl, alkoxy, alkoxycarbonyl, carboxy, or —(CH₂)ₓ—NR²⁹R³⁰, where R²⁹ and R³⁰ are each independently selected from H and alkyl,
or R²⁷ and R²⁸ combine to form a 5- or 6-membered ring, optionally containing one or more additional heteroatoms, optionally containing one or more degrees of unsaturation, and optionally substituted one or more times with alkyl, hydroxy, carboxy, acyl, alkoxy, or halogen,
or —(O)_{y}—(CH₂)ₓ—R³¹ where R³¹ is hydroxy, alkoxy, haloalkyl, aryl optionally substituted with halogen, or —NR²⁷R²⁸, where R²⁷ and R²⁸ are as defined above;
wherein for each occurrence, x independently is 0, 1, 2, or 3;
wherein for each occurrence, y independently is 0 or 1; and
and R² is heteroaryl substituted one or more times with alkyl, alkoxy, halogen, haloalkyl, haloalkoxy, nitro, or —NR³¹R³², wherein R³¹ and R³² are each independently selected from H and alkyl.

2. The compound of claim 1 wherein R² is pyridinyl.

3. The compound of claim 2 wherein R² is pyridinyl substituted with alkoxy.

4-7. (canceled)

8. The compound of claim 1 wherein R² is thiazolyl.

9. The compound of claim 1 wherein R² is benzimidazolyl.

10. (canceled)

11. A pharmaceutical composition comprising:
a therapeutically effective amount of a compound as claimed in claim 1.

12. The pharmaceutical composition of claim 11 further comprising:
one or more of pharmaceutically acceptable carriers, diluents, or excipients.

13. A method of treating a disorder in a mammal, said disorder being characterized by misregulation of one or more protein kinase comprising:
administering to said mammal a therapeutically effective amount of a compound as claimed in claim 1.

14. (canceled)

15. The method of claim 13 wherein the kinase is GSK3.

16-19. (canceled)

20. A compound according to claim 1 with reference to any of the Examples.

21. A compound of Formula (II): 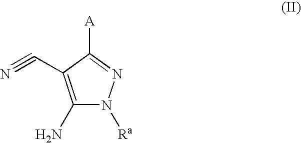
including salts, solvates, and pharmaceutically functional derivatives thereof, where A is H, alkyl, or aryl; and
R^{a} is heteroaryl substituted one or more times with alkyl, alkoxy, halogen, haloalkyl, haloalkoxy, nitro, or —NR^{b}R^{c}, wherein R^{b} and R^{c} are each independently selected from H and alkyl.

22. (canceled)

23. The compound of claim 21 wherein R^{a} is selected from 2-pyridyl, thiazolyl, or benzimidazolyl.

24. The compound of claim 21 wherein R^{a} is 2-pyridyl substituted with alkoxy.

25. (canceled)

26. A compound of formula (III) 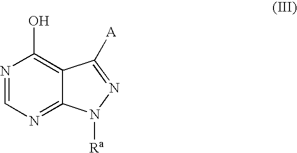
including salts, solvates, and pharmaceutically functional derivatives thereof, where A is H, alkyl, or aryl; and
R^{a} is heteroaryl substituted one or more times with alkyl, alkoxy, halogen, haloalkyl, haloalkoxy, nitro, or —NR^{b}R^{c}, wherein R^{b} and R^{c} are each independently selected from H and alkyl.

27. (canceled)

28. The compound of claim 26 wherein R^{a} is selected from pyridyl, thiazolyl, or benzimidazolyl.

29. The compound of claim 26 wherein R^{a} is pyridyl substituted with alkoxy.

30. (canceled)

31. A compound of formula (IV) 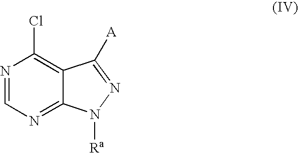
including salts, solvates, and pharmaceutically functional derivatives thereof, where A is H, alkyl, or aryl; and
R^{a} is heteroaryl substituted one or more times with alkyl, alkoxy, halogen, haloalkyl, haloalkoxy, nitro, or —NR^{b}R^{c}, wherein R^{b} band R^{c} are each independently selected from H and alkyl.

32. (canceled)

33. The compound of claim 31 wherein R^{a} is selected from pyridyl, thiazolyl, or benzimidazolyl.

34. The compound of claim 31 wherein R^{a} is pyridyl substituted with alkoxy.

35. (canceled)

36. A compound of formula (V) 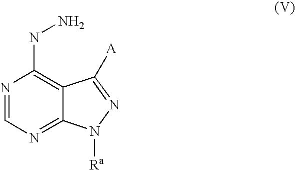
including salts, solvates, and pharmaceutically functional derivatives thereof, where A is H, alkyl, or aryl; and
R^{a} is heteroaryl substituted one or more times with alkyl, alkoxy, halogen, haloalkyl, haloalkoxy, nitro, or —NR^{b}R^{c}, wherein R^{b} and R^{c} are each independently selected from H and alkyl.

37. (canceled)

38. The compound of claim 36 wherein R^{a} is selected from pyridyl, thiazolyl, or benzimidazolyl.

39. The compound of claim 36 wherein R^{a} is pyridyl substituted with alkoxy.

40. (canceled)
